(19) United States
(12) Patent Application Publication
(10) Pub. No.: US 2012/0115793 A1
(43) Pub. Date: May 10, 2012

(54) PEPTIDOMIMETIC MACROCYCLES

(75) Inventors: Huw M. Nash, Concord, MA (US); Rosana Kapeller-Libermann, Chesnut Hill, MA (US); Jiawen Han, Newton, MA (US); Tomi K. Sawyer, Southborough, MA (US); Justin Noehre, Cambridge, MA (US)

(73) Assignee: Alleron Therapeutics, Inc, Cambridge, MA (US)

(21) Appl. No.: 13/120,386

(22) PCT Filed: Sep. 22, 2009
(86) PCT No.: PCT/US09/57928

§ 371 Date: Dec. 22, 2011

Related U.S. Application Data
(62) Division of application No. 61/099,151, filed on Sep. 22, 2008.

(51) Int. Cl. A61K 38/12; C07K 14/00; A61P 35/00; C07K 7/56

(52) U.S. Cl. 514/19.9; 530/317; 514/21.1

(57) ABSTRACT

The present invention provides novel peptidomimetic macrocycles and methods of using such macrocycles for the treatment of disease.

### CROSS-REFERENCE

This application claims the benefit of U.S. Provisional Application No. 61/099,151, filed Sep. 22, 2008, which application is incorporated herein by reference.

### BACKGROUND OF THE INVENTION

Notch receptors are transmembrane receptors that are involved in a variety of important signaling pathways. Vertebrates possess four different notch receptors, referred to as Notch1 to Notch4. Notch receptors are key regulators of cell proliferation, stem cells and stem cell niche maintenance, cell fate acquisition, cell differentiation, and cell death. Notch is a phylogenetically conserved transmembrane receptor that is required for many aspects of animal development. Upon ligand stimulation, a fragment of Notch is released proteolytically and enters the nucleus to form a complex with the DNA-binding protein CSL (CBF1/Suppressor of Hairless/Lag1) and activate transcription of Notch-CSL target genes. Mutations in human Notch 1 are commonly found in human T cell acute lymphoblastic leukemias (T-ALL) and abnormalities in Notch signaling are also implicated in genesis and progression of other types of cancers including breast cancer, melonoma, and colon cancer. The Notch signaling pathway is complex. When an appropriate ligand binds to Notch a proteolytic event occurs which allows a portion of the Notch receptor called ICN to enter the cell nucleus where it interacts with CSL, a transcription factor that binds DNA, and a protein that is a member of the Mastermind-like (MAML) family. The assembled complex can activate transcription of certain genes. It is known that certain fragments of MAML (e.g., within amino acids 13-74 of human MAML-I) can act to interfere with Notch activation of transcription.

Currently there are no small molecule inhibitors of the Notch/CSL/MAML ternary complex. γ-secretase inhibitors (GSIs) can block Notch receptor signaling in vitro, however, the current peptide therapeutics are not specific to Notch 1 and may have the issues of toxicity and development of drug resistance similar to GSIs and Gleevec, the latter of which is a specific inhibitor of a number of tyrosine kinase enzymes. Thus, there is a strong need for development of therapeutics e.g. inhibitors that selectively target Notch, e.g. Notch 1, and can induce killing rather than cell cycle arrest of the target cells. Such therapeutics may be used in the treatment of a variety of cancers including but not limited to T cell acute lymphoblastic leukemias (T-ALL) and may restore sensitivity of T-ALL to steroid therapy.

### SUMMARY OF THE INVENTION

In one aspect, the present invention provides a peptidomimetic macrocycle comprising an amino acid sequence which is at least about 60%, 80%, 90%, or 95% identical to an amino acid sequence chosen from the group consisting of the amino acid sequences in Table 1. Alternatively, an amino acid sequence of said peptidomimetic macrocycle is chosen from the group consisting of the amino acid sequences in Table 1. In some embodiments, the peptidomimetic macrocycle comprises a helix, such as an α-helix. In other embodiments, the peptidomimetic macrocycle comprises an α,α-disubstituted amino acid. A peptidomimetic macrocycle of the invention may comprise a crosslinker linking the α-positions of at least two amino acids. At least one of said two amino acids may be an α,α-disubstituted amino acid.

In some embodiments, the peptidomimetic macrocycle has the formula:

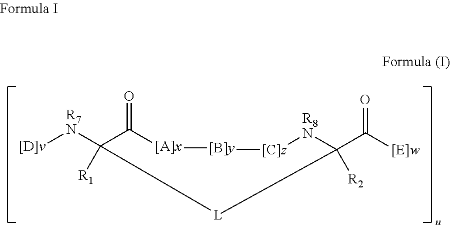

wherein:

each A, C, D, and E is independently a natural or non-natural amino acid;

B is a natural or non-natural amino acid, amino acid analog;

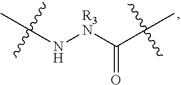

### [—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula -L₁-L₂-;

L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

R₅ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

v and w are independently integers from 1-1000;

u, x, y and z are independently integers from 0-10; and

n is an integer from 1-5.

In other embodiments, the peptidomimetic macrocycle may comprise a crosslinker linking a backbone amino group of a first amino acid to a second amino acid within the peptidomimetic macrocycle. For example, the invention provides peptidomimetic macrocycles of the formula (IV) or (IVa):

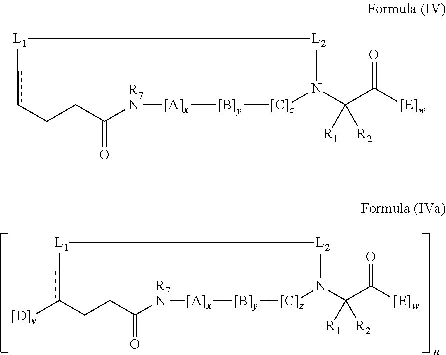

wherein:

each A, C, D, and E is independently a natural or non-natural amino acid;

B is a natural or non-natural amino acid, amino acid analog

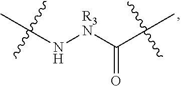

### [—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅;

v and w are independently integers from 1-1000;

u, x, y and z are independently integers from 0-10; and

n is an integer from 1-5.

Additionally, the invention provides a method of treating cancer in a subject comprising administering to the subject a peptidomimetic macrocycle of the invention. Also provided is a method of modulating the activity of Notch in a subject comprising administering to the subject a peptidomimetic macrocycle of the invention, or a method of antagonizing the interaction between MAML and Notch or CSL proteins in a subject comprising administering to the subject such a peptidomimetic macrocycle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a possible binding mode of an hMAML peptidomimetic macrocycle precursor of the invention to Notch/CSL/DNA complex.

FIGS. 2 and 3 illustrate possible binding modes of hMAML peptidomimetic macrocycles of the invention to Notch/CSL/DNA complex.

FIG. 4 shows exemplary peptidomimetic macrocycles of the invention.

### DETAILED DESCRIPTION OF THE INVENTION

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analog) and a second naturally-occurring or non-naturally-occurring amino acid residue (or analog) within the same molecule. Peptidomimetic macrocycle include embodiments where the macrocycle-forming linker connects the α carbon of the first amino acid residue (or analog) to the α carbon of the second amino acid residue (or analog). The peptidomimetic macrocycles optionally include one or more non-peptide bonds between one or more amino acid residues and/or amino acid analog residues, and optionally include one or more non-naturally-occurring amino acid residues or amino acid analog residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild-type sequence corresponding to the macrocycle.

As used herein, the term “stability” refers to the maintenance of a defined secondary structure in solution by a peptidomimetic macrocycle of the invention as measured by circular dichroism, NMR or another biophysical measure, or resistance to proteolytic degradation in vitro or in vivo. Non-limiting examples of secondary structures contemplated in this invention are α-helices, β-turns, and β-pleated sheets.

As used herein, the term “helical stability” refers to the maintenance of α helical structure by a peptidomimetic macrocycle of the invention as measured by circular dichroism or NMR. For example, in some embodiments, the peptidomimetic macrocycles of the invention exhibit at least a 1.25, 1.5, 1.75 or 2-fold increase in α-helicity as determined by circular dichroism compared to a corresponding uncrosslinked macrocycle.

The term “α-amino acid” or simply “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The term “amino acid analog” or “non-natural amino acid” refers to a molecule which is structurally similar to an amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine, or substitution or the carboxy group with an ester).

A “non-essential” amino acid residue is a residue that can be altered from the wild-type sequence of a polypeptide (e.g., a BH3 domain or the p53 MDM2 binding domain) without abolishing or substantially altering its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a BH3 polypeptide, for example, is preferably replaced with another amino acid residue from the same side chain family. Other examples of acceptable substitutions are substitutions based on isosteric considerations (e.g. norleucine for methionine) or other properties (e.g. 2-thienylalanine for phenylalanine).

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

The symbol “ [symbol] ” when used as part of a molecular structure refers to a single bond or a trans or cis double bond.

The term “amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,α di-substituted amino acid).

The term “α,α di-substituted amino” acid refers to a molecule or moiety containing both an amino group and a carboxyl group bound to a carbon (the α-carbon) that is attached to two natural or non-natural amino acid side chains.

The term “polypeptide” encompasses two or more naturally or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “macrocyclization reagent” or “macrocycle-forming reagent” as used herein refers to any reagent which may be used to prepare a peptidomimetic macrocycle of the invention by mediating the reaction between two reactive groups. Reactive groups may be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents may additionally include, for example, Ru reagents known in the art such as Cp*RuCl(PPh₃)₂, [Cp*RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Ace. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms in it.

The term “alkylene” refers to a divalent alkyl (i.e., —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms in it. The term “lower alkenyl” refers to a C₂-C₆ alkenyl chain. In the absence of any numerical designation, “alkenyl” is a chain (straight or branched) having 2 to 20 (inclusive) carbon atoms in it.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms in it. The term “lower alkynyl” refers to a C₂-C₆ alkynyl chain. In the absence of any numerical designation, “alkynyl” is a chain (straight or branched) having 2 to 20 (inclusive) carbon atoms in it.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH2-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl,

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH3, and —CH₂—NH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a—COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds of this invention contain one or more asymmetric centers and thus occur as racemates and racemic mixtures, single enantiomers, individual diastereomers and diastereomeric mixtures. All such isomeric forms of these compounds are included in the present invention unless expressly provided otherwise. In some embodiments, the compounds of this invention are also represented in multiple tautomeric forms, in such instances, the invention includes all tautomeric forms of the compounds described herein (e.g., if alkylation of a ring system results in alkylation at multiple sites, the invention includes all such reaction products). All such isomeric forms of such compounds are included in the present invention unless expressly provided otherwise. All crystal forms of the compounds described herein are included in the present invention unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the invention may be practiced with the variable equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values and 2 if the variable is inherently continuous.

As used herein, unless specifically indicated otherwise, the word “or” is used in the inclusive sense of “and/or” and not the exclusive sense of “either/or.”

The term “on average” represents the mean value derived from performing at least three independent replicates for each data point.

The term “biological activity” encompasses structural and functional properties of a macrocycle of the invention. Biological activity is, for example, structural stability, alpha-helicity, affinity for a target, resistance to proteolytic degradation, cell penetrability, intracellular stability, in vivo stability, or any combination thereof.

The details of one or more particular embodiments of the invention are set forth in the accompanying drawings and the description below. Other features, objects, and advantages of the invention will be apparent from the description and drawings, and from the claims.

In some embodiments, the peptide sequence is derived from a protein of the Mastermind-like (MAML) family that binds to the Notch/CSL/DNA complex. The MAML (mastermind-like) proteins are a family of three cotranscriptional regulators that are essential for Notch signaling, a pathway critical for cell fate determination. The distinct tissue distributions of MAML proteins and differential activities in cooperating with various Notch receptors suggest that they have unique roles. For example, mice with a targeted disruption of the MAML-1 gene have severe muscular dystrophy (Shen H. et. al., Genes & development 2006, vol. 20). In vitro, Maml1-null embryonic fibroblasts fail to undergo MyoD-induced myogenic differentiation, further suggesting that MAML1 is required for muscle development. Moreover, MAML1 interacts with MEF2C (myocyte enhancer factor 2C), functioning as its potent cotranscriptional regulator. However, MAML 1's promyogenic effects are completely blocked upon activation of Notch signaling, which is associated with recruitment of MAML1 away from MEF2C to the Notch transcriptional complex. Mechanistically, MAML1 appears to mediate cross-talk between Notch and MEF2 to influence myogenic differentiation.

The Notch receptor is a single-pass transmembrane receptor protein. It is a hetero-oligomer composed of a large extracellular portion which associates in a calcium dependent, non-covalent interaction with a smaller piece of the Notch protein composed of a short extracellular region, a single transmembrane-pass, and a small intracellular region (Annika E. et. al. 2002 Molecular and Cellular Biology 22 (22): 7812-7819). Ligand proteins binding to the extracellular domain of Notch receptor induce proteolytic cleavage and release of the intracellular domain, which enters the cell nucleus to alter gene expression (Franz Oswald; et. al. 2001 Molecular and Cellular Biology 21 (22): 7761-7774).

Maturation of the Notch receptor involves cleavage at the prospective extracellular side during intracellular trafficking in the Golgi complex. This results in a bipartite protein, composed of a large extracellular domain linked to the smaller transmembrane and intracellular domain. Binding of ligand promotes two proteolytic processing events; as a result of proteolysis, the intracellular domain is liberated and can enter the nucleus to engage other DNA-binding proteins and regulate gene expression. Notch and most of its ligands are transmembrane proteins, so the cells expressing the ligands typically need to be adjacent to the Notch expressing cell for signaling to occur. The Notch ligands are also single-pass transmembrane proteins and are members of the DSL (Delta/Serrate/LAG-2) family of proteins. In mammals, the ligands are Delta-like and Jagged. In mammals there are multiple Delta-like and Jagged ligands, as well as possibly a variety of other ligands, such as F3/contactin (Eric C. Lai 2004 Development 131).

The Notch signaling pathway is important for cell-cell communication, which involves gene regulation mechanisms that control multiple cell differentiation processes during embryonic and adult life. Notch signaling also plays important role in processes including but not limited to: neuronal function and development, stabilizing arterial endothelial fate and angiogenesis, regulating crucial cell communication events between endocardium and myocardium during both the formation of the valve primordial and ventricular development and differentiation, cardiac valve homeostasis as well as implications in other human disorders involving the cardiovascular system, timely cell lineage specification of both endocrine and exocrine pancreas, influencing binary fate decisions of cells that must choose between the secretory and absorptive lineages in the gut, expanding the HSC compartment during bone development and participation in commitment to the osteoblastic lineage suggesting a potential therapeutic role for Notch in bone regeneration and osteoporosis, regulating cell-fate decision in mammary gland at several distinct development stages, and possibly some non-nuclear mechanisms, such as controlling the actin cytoskeleton through the tyrosine kinase Abl (Gaiano N; Fishell G (March 2002) Annual Reviews of Neuroscience 25: 471. Bolos V; Grego-Bessa J, J, de la Pompa J L. (April 2007) Endocrine Reviews 28: 339. Zhao-Jun Liu; et al (January 2003). Molecular and Cellular Biology 23 (1): 14-25. Joaquín Grego-Bessa et al (March 2007). Developmental Cell 12 (3): 415-429. L. Charles Murtaugh et. al. 2003 Proc Natl Acad Sci USA. 100 (25): 14920-5. Guy R. Sander; Barry C. Powell 2004 Journal of Histochemistry and Cytochemistry 52 (4): 509-516. Masuhiro Nobta et al. 2005 J. Biol. Chem. 280 (16): 15842-48. Dontu, G. et. al. 2004 Breast Cancer Res. 6; Eric C. Lai 2004 Development 131).

Notch signaling is dysregulated in many cancers, and faulty Notch signaling is implicated in many diseases including but not limited to T-ALL (T-cell acute lymphoblastic leukemia) (Sharma V. M. et. al. 2007 Cell Cycle 6 (8): 927-930), CADASIL (Cerebral Autosomal Dominant Arteriopathy with Sub-cortical Infarcts and Leukoencephalopathy), MS (Multiple Sclerosis), Tetralogy of Fallot, Alagille syndrome, and myriad other disease states.

Gain-of-function mutations in Notch 1 are the most common acquired genetic lesions in T-ALL accounting for approximately 60% of lesions in T-ALL. There are two mutational hot spots that contribute to the development of T-ALL. First, mutation at the heterodimerization domain leads to ligand-independent cleavage of Notch resulting in a constitutive release of the intracellular portion of the Notch receptor (ICN) (Weng et. al. Science Vol 306,2004). The heterodimerization (HD) domain responsible for stable subunit association consists of a 103 amino acid region of the extracellular Notch and a 65 amino acid region in transmembrane subunits (NTM). Physiologic activation of NOTCH receptors occurs when ligands of the Delta-Senate-Lag2 (DSL) family bind to the extracellular subunit and initiate a cascade of proteolytic cleavages in the NTM subunit. The final cleavage, catalyzed by γ-secretase, generates intracellular Notch (ICN), which translocates to the nucleus and forms a large transcriptional activation complex that includes proteins of the MAML family. The HD domain mutations enhance γ-secretase cleavage and increase the rate of production of ICN1. The second mutation in Notch is the deletion of C-terminal PEST sequence. Cellular levels of ICN are determined by the net effects of the rates of production and destruction of the protein. The SCF-FBW7 ubiquitin ligase plays a critical role in ICN degradation that is dependent on an intact PEST domain of Notch. Deletion of C-terminal PEST sequence leads to stabilization of ICN by increasing the half life of ICN1 (Gupta-Rossi et. al. J Biol. Chem. 276, 2001). Aberrant Notch activation in T cells leads to increased c-myc expression, dysregulation of cell metabolism and suppression of the tumor-suppressor p53 function, all of which contribute to the development of cancer.

The Notch extracellular domain is composed primarily of small cysteine knot motifs called EGF-like repeats (Bing Ma, et. al. 2006 Glycobiology 16 (12). Notch 1 for example has 36 of these repeats. Each EGF-like repeat is approximately 40 amino acids, and its structure is defined largely by six conserved cysteine residues that form three conserved disulfide bonds. Each EGF-like repeat can be modified by O-linked glycans at specific sites. An O-glucose sugar may be added between the first and second conserved cysteine, and an O-fucose may be added between the second and third conserved cysteine. These sugars are added by an as yet unidentified O-glucosyltransferase, and GDP-fucose Protein O-fucosyltransferase 1 (POFUT1) respectively. The addition of O-fucose by POFUT1 is absolutely necessary for Notch function, and without the enzyme to add O-fucose, all Notch proteins fail to function properly. The O-glucose on Notch can be further elongated to a trisaccharide with the addition of two xylose sugars by xylosyltransferases, and the O-fucose can be elongated to a tetrasaccharide by the ordered addition of an N-acetylglucosamine (GlcNAc) sugar by an N-Acetylglucosaminyltransferase called Fringe, the addition of a galactose by a galactosyltransferase, and the addition of a sialic acid by a sialyltransferase (Lu L.; Stanley P., 2006 Methods in Enzymology 417: 127-136). In mammals there are three Fringe GlcNAc-transferases, named Lunatic Fringe, Manic Fringe, and Radical Fringe. These enzymes are responsible for the “Fringe Effect” on Notch signaling. If Fringe adds a GlcNAc to the O-fucose sugar, then the subsequent addition of a galactose and sialic acid will occur. In the presence of this tetrasaccharide, Notch signals strongly when it interacts with the Delta ligand, but has markedly inhibited signaling when interacting with the Jagged ligand. Once the Notch extracellular domain interacts with a ligand, an ADAM-family metalloprotease called TACE (Tumor Necrosis Factor Alpha Converting Enzyme) cleaves the Notch protein just outside the membrane (Brou C., et. al. 2000 Molecular Cell 5 (2): 207-16). This releases the extracellular portion of Notch, which continues to interact with the ligand. The ligand plus the Notch extracellular domain is then endocytosed by the ligand-expressing cell. After this first cleavage, an enzyme called γ-secretase cleaves the remaining part of the Notch protein just inside the inner leaflet of the cell membrane of the Notch-expressing cell. This releases the intracellular domain of the Notch protein, which then moves to the nucleus where it can regulate gene expression by activating the transcription factor CSL (Eric C. Lai 2004 Development 131). Other proteins also participate in the intracellular portion of the Notch signaling cascade.

### Structure of the Notch/CSL/MAML Ternary Complex

Once Notch translocates to the nucleus, it engages CSL converting it from a transcriptional repressor to an activator (Mumm and Kopan, 2000). In the absence of a signal, CSL represses transcription of Notch target genes by recruiting corepressor proteins to form a multiprotein transcriptional repressor complex (Kao et al., 1998 and Hsieh et al., 1999). In the presence of a signal, Notch ICN binding to CSL displaces corepressors from CSL (Kao et al., 1998 and Zhou et al., 2000), leading to the binding of the transcriptional coactivator MAML to the complex (Petcherski and Kimble, 2000 and Wu et al., 2002). Activation of transcription occurs by the recruitment of general transcription factors to the CSL-Notch ICN-MAML ternary complex (Kurooka and Honjo, 2000, Fryer et al., 2002 and Wallberg et al., 2002).

CSL is composed of three integrated domains: the N-terminal domain (NTD), the β trefoil domain (BTD), and the C-terminal domain (CTD). The NTD and CTD share structural similarities with the Rel-homology-region family of transcription factors. The NTD of CSL interacts with the major groove of DNA in a similar manner to Rel proteins; however, in contrast to the Rel family, the BTD contributes to minor groove DNA binding in a novel manner, and the CTD does not interact with the DNA at all. The CSL-DNA structural determination further reveals that the BTD of CSL has an atypical β trefoil fold, which results in a large exposed hydrophobic surface with a distinctive pocket on the BTD, providing a compelling site for interaction with a hydrophobic ligand.

Notch ICN consists of at least three domains, the membrane-proximal RAM (RBP-jκ-associated molecule) domain, followed by seven consecutive ankyrin repeats (ANK) and a C-terminal PEST sequence. In vitro, Notch ICN interacts strongly with CSL through its RAM domain (Tamura et al., 1995) but only weakly with its ankyrin repeats (Kato et al., 1997). However, the ankyrin repeats are required for formation of the CSL-Notch ICN-MAML ternary complex (Nam et al., 2003) and transcriptional activation (Jarriault et al., 1995). The CSL-RAM domain interaction is necessary for signaling in vivo.

Mastermind (MAML) is a glutamine-rich transcriptional coactivator protein that is localized to the nucleus. A short, approximately 75-residue, N-terminal domain of MAML is required for binding to the CSL-Notch complex, which additionally requires the three conserved domains of CSL (NTD, BTD, and CTD) and the ANK domain of Notch (Nam et al., 2003). Mastermind has dual roles of both activating Notch target gene transcription through the direct binding of CBP/p300 and promoting hyperphosphorylation and degradation of Notch ICN (Wallberg et al., 2002 and Fryer et al., 2004).

Crystal structure of the ternary complex of CSL, Notch, and MAML bound to a target DNA reveals that Notch ICN interacts with CSL through its RAM and ankyrin repeats domains binding to the BTD and CTD of CSL, respectively. RAM binding to BTD alters the conformation of a conserved loop within the BTD, which has functional implications for corepressor displacement from CSL. MAML interacts with the ankyrin repeats of Notch and the CTD of CSL, forming a three-way protein interface with additional important contacts made by MAML and the NTD of CSL. More specifically, the structure of MAML is composed of two long α helices with a distinct bend centered on Pro86 and an N-terminal extension that is in an extended conformation. The N-terminal helix and extension of MAML interact with ANK of Notch and the CTD of CSL, whereas the C-terminal MAML helix interacts with a concave surface on the NTD of CSL formed by its β sheet structure. The MAML-1 polypeptide “motif” in the Notch transcriptional activation complex includes a 52 residue helix, much longer than the typical recognition motif. By recognizing parts of ANK of Notch and CSL at alternating surfaces along the long axis of the ANK:CSL protein-protein interface, MAML-1 ensures binding to the Notch:CSL complex with high affinity, in the absence of tight binding to either protein alone. Further stringency in recognition is achieved by requiring the MAML-1 sequence to fold into a relatively rigid helical conformation to form a productive complex, because the MAML-1 polypeptide is not folded until bound. Formation of the CSL-Notch-MAML ternary complex induces a large structural change in the orientation of the domains within CSL while maintaining similar DNA binding contacts and specificity (Wilson J, et. al. Cell 124, 2006). A model for stepwise assembly of the core of the Notch transcriptional activation complex has been proposed in which intracellular Notch is initially recruited to the CSL:DNA complex by the RAM sequence of Notch, which has high affinity for the β-trefoil domain of CSL. The ANK domain of Notch then docks against the Rel-homology portion of CSL to create a high-affinity binding site for MAML-1. In the model, transient association of ANK of Notch with the Rel-homology domain of CSL becomes clamped by MAML-1 binding (Nam Y. et al. Cell 124, 2006).

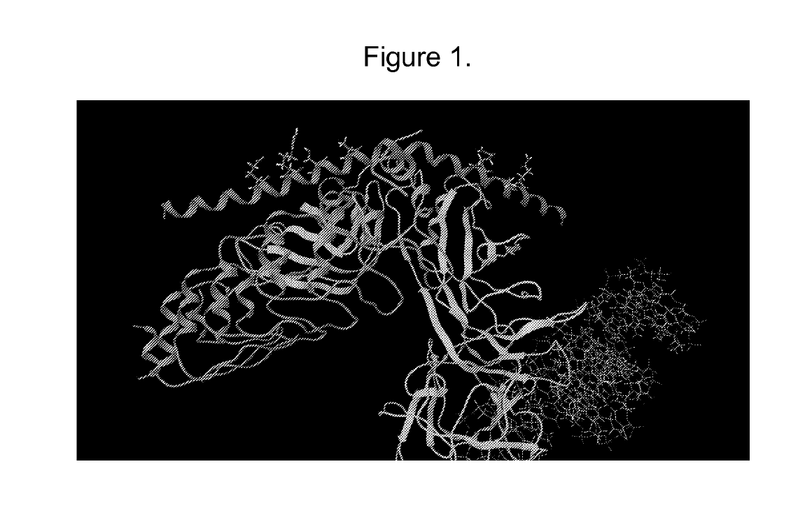

Analysis of conserved residues among MAML-1, 2 and 3; analysis of the predicted interaction between MAML and Notch; and analysis of predicted alpha-helical regions have led to the identification amino acids that might be replaced to provide a cross-link without significantly inhibiting binding to Notch. As shown in FIGS. 1-3, for human MAML, the sequence of the residues 21-42 that may be used in the present invention for binding to Notch/CSL is ERLRRRIELCRRHHSTCEARYE. Solvent exposed side-chains available for cross-linking are underlined. Highly conserved amino acids among MAML polypeptides and those thought to be important in protein-protein interactions based on X-ray crystallographic are preferably not replaced.

A non-limiting exemplary list of suitable MAML-Notch/CSL peptides for use in the present invention is given below:

TABLE 1

| MAML sequences suitable for synthesis of peptidomimetic macrocycles | | | | | | | | | | | | | | | | | | | | | | | | | Design | |
|---|---|---|---|---|---|---|---|---|---|---|---|---|---|---|---|---|---|---|---|---|---|---|---|---|---|---|
| (bold = critical residue; X = cross-linked amino acid) | | | | | | | | | | | | | | | | | | | | | | | | | Notes | |
| Ac | | E | R | L | R | R | R | I | E | L | C | R | R | H | H | S | T | C | E | A | R | Y | E | —NH2 | wild-type | |
| - | | | | | | | | | | | | | | | | | | | | | | | | | | |
| Ac | | E | R | L | R | R | R | I | E | L | C | R | R | H | H | X | T | C | E | X | R | Y | E | —NH2 | i-->i + 4x-link | |
| - | | | | | | | | | | | | | | | | | | | | | | | | | | |
| Ac | | E | R | L | R | R | R | I | E | L | C | R | X | H | H | S | X | C | E | A | R | Y | E | —NH2 | i-->i + 4x-link | |
| - | | | | | | | | | | | | | | | | | | | | | | | | | | |
| Ac | | E | R | L | R | R | R | I | X | L | C | R | X | H | H | S | T | C | E | A | R | Y | E | —NH2 | i-->i + 4x-link | |
| - | | | | | | | | | | | | | | | | | | | | | | | | | | |
| Ac | | E | R | L | R | R | X | I | E | L | X | R | R | H | H | S | T | C | E | A | R | Y | E | —NH2 | i-->i + 4x-link | |
| - | | | | | | | | | | | | | | | | | | | | | | | | | | |
| Ac | | E | R | L | X | R | R | I | X | L | C | R | R | H | H | S | T | C | E | A | R | Y | E | —NH2 | i-->i + 4x-link | |
| - | | | | | | | | | | | | | | | | | | | | | | | | | | |
| Ac | | E | R | L | R | R | R | I | E | L | C | R | X | H | H | S | T | C | E | X | R | Y | E | —NH2 | i-->i +7x-link | |
| - | | | | | | | | | | | | | | | | | | | | | | | | | | |
| Ac | | E | R | L | R | R | R | I | X | L | C | R | R | H | H | X | T | C | E | A | R | Y | E | —NH2 | i-->i + 7x-link | |
| - | | | | | | | | | | | | | | | | | | | | | | | | | | |
| Ac | | E | R | L | R | R | R | I | X | L | C | R | X | H | H | S | T | C | E | X | R | Y | E | —NH2 | i-->i + 4, | |
| - | | | | | | | | | | | | | | | | | | | | | | | | | i-->i + 7x-link | |
| Ac | | E | R | L | X | R | R | I | X | L | C | R | R | H | H | X | T | C | E | A | R | Y | E | —NH2 | i-->i + 4, | |
| - | | | | | | | | | | | | | | | | | | | | | | | | | i-->i + 7x-link | |
| Ac | X | E | R | X | R | R | R | I | E | L | C | R | R | H | H | S | T | C | E | A | R | Y | E | —NH2 | Arorax-link | |
| - | | | | | | | | | | | | | | | | | | | | | | | | | | |
| Ac | X | E | R | X | R | R | R | I | E | L | C | R | R | H | H | X | T | C | E | X | R | Y | E | —NH2 | Arora, i-->l + 4 | |
| - | | | | | | | | | | | | | | | | | | | | | | | | | | |
| Ac | X | E | R | X | R | R | R | I | E | L | C | R | X | H | H | S | X | C | E | A | R | Y | E | —NH2 | Arora, i-->1 + 4 | |
| - | | | | | | | | | | | | | | | | | | | | | | | | | | |
| Ac | | E | R | L | R | R | R | I | E | L | C | R | R | H | H | S | T | | | | | | | —NH2 | wild-type | |
| - | | | | | | | | | | | | | | | | | | | | | | | | | | |
| Ac | | E | R | L | R | R | R | I | X | L | C | R | X | H | H | S | T | | | | | | | —NH2 | i-->i + 4x-link | |
| - | | | | | | | | | | | | | | | | | | | | | | | | | | |
| Ac | | E | R | L | R | R | X | I | E | L | X | R | R | H | H | S | T | | | | | | | —NH2 | i-->i + 4x-Iink | |
| - | | | | | | | | | | | | | | | | | | | | | | | | | | |
| Ac | | E | R | L | X | R | R | I | X | L | C | R | R | H | H | S | T | | | | | | | —NH2 | i-->i + 4x-link | |
| - | | | | | | | | | | | | | | | | | | | | | | | | | | |
| Ac | | E | R | L | R | R | R | I | X | L | C | R | R | H | H | X | T | | | | | | | —NH2 | i-->i + 7x-link | |
| - | | | | | | | | | | | | | | | | | | | | | | | | | | |
| Ac | | E | R | L | R | R | X | I | E | L | C | R | X | H | H | S | T | | | | | | | —NH2 | i-->i + 7x-link | |
| - | | | | | | | | | | | | | | | | | | | | | | | | | | |
| Ac | | E | R | L | X | R | R | I | X | L | C | R | R | H | H | X | T | | | | | | | —NH2 | i-->i + 4, | |
| - | | | | | | | | | | | | | | | | | | | | | | | | | i-->i + 7x-link | |
| Ac | X | E | R | L | X | R | R | I | E | L | C | R | R | H | H | S | T | | | | | | | —NH2 | Arora | |
| - | | | | | | | | | | | | | | | | | | | | | | | | | | |
| Ac | | | | | | R | R | I | E | L | C | K | R | H | H | S | T | C | E | A | R | Y | E | —NH2 | wild-type | |
| - | | | | | | | | | | | | | | | | | | | | | | | | | | |
| Ac | | | | | | R | R | I | E | L | C | R | R | H | H | X | T | C | E | X | R | Y | E | —NH2 | i->i + 4x-link | |
| - | | | | | | | | | | | | | | | | | | | | | | | | | | |
| Ac | | | | | | R | R | I | E | L | C | R | X | H | H | S | X | C | E | A | R | Y | E | —NH2 | i->i + 4x-link | |
| - | | | | | | | | | | | | | | | | | | | | | | | | | | |
| Ac | | | | | | R | R | I | X | L | C | R | X | H | H | S | T | C | E | A | R | Y | E | —NH2 | i->i + 4x-link | |
| - | | | | | | | | | | | | | | | | | | | | | | | | | | |
| Ac | | | | | | R | X | I | E | L | X | R | R | H | H | S | T | C | E | A | R | Y | E | —NH2 | i->i + 4x-link | |
| - | | | | | | | | | | | | | | | | | | | | | | | | | | |
| Ac | | | | | | R | R | I | X | L | C | R | X | H | H | S | T | C | E | X | R | Y | E | —NH2 | i->i + 4, | |
| - | | | | | | | | | | | | | | | | | | | | | | | | | i->i + 7x-link | |
| MAML peptidomimetic macrocycles (bold = mutation; $ = S5 olefin | | | | | | | | | | | | | | | | | | | | | | | | | | Charge at |
| amino acid; $r8 = R8 olefin amino acid) | | | | | | | | | | | | | | | | | | | | | | | | | | pH 7.4 |
| Ac | | E | R | L | R | R | R | I | $ | L | C | R | $ | H | H | S | T | | | | | | | —NH2 | | 4 |
| - | | | | | | | | | | | | | | | | | | | | | | | | | | |
| Ac | | E | R | L | A | R | A | I | $ | L | C | R | $ | H | H | S | T | | | | | | | —NH2 | | 2 |
| - | | | | | | | | | | | | | | | | | | | | | | | | | | |
| Ac | | E | R | L | R | R | R | I | $ | L | A | R | $ | H | H | S | T | | | | | | | —NH2 | | 4 |
| - | | | | | | | | | | | | | | | | | | | | | | | | | | |
| Ac | | E | R | L | A | R | A | I | $ | L | A | R | $ | H | H | S | T | | | | | | | —NH2 | | 2 |
| - | | | | | | | | | | | | | | | | | | | | | | | | | | |
| Ac | | E | R | L | R | R | $ | I | E | L | $ | R | A | H | H | S | T | | | | | | | —NH2 | | 2 |
| - | | | | | | | | | | | | | | | | | | | | | | | | | | |
| Ac | | E | R | L | $ | R | R | I | $ | L | C | R | R | H | H | S | T | | | | | | | —NH2 | | 4 |
| - | | | | | | | | | | | | | | | | | | | | | | | | | | |
| Ac | | E | R | L | $ | R | R | I | $ | L | C | R | A | H | H | S | T | | | | | | | —NH2 | | 3 |
| - | | | | | | | | | | | | | | | | | | | | | | | | | | |
| Ac | | E | R | L | $ | R | R | I | $ | L | A | R | A | H | H | S | T | | | | | | | —NH2 | | 3 |
| - | | | | | | | | | | | | | | | | | | | | | | | | | | |
| Ac | | E | R | L | R | R | A | I | $r8 | L | C | R | A | H | H | $ | T | | | | | | | —NH2 | | 3 |
| - | | | | | | | | | | | | | | | | | | | | | | | | | | |
| Ac | | E | R | L | R | R | A | I | $r8 | L | A | R | A | H | H | $ | T | | | | | | | —NH2 | | 3 |
| - | | | | | | | | | | | | | | | | | | | | | | | | | | |
| Ac | | | | | | R | R | I | E | L | C | R | R | H | H | $ | T | C | E | $ | R | Y | E | —NH2 | | 2 |
| - | | | | | | | | | | | | | | | | | | | | | | | | | | |
| Ac | | | | | | R | A | I | E | L | C | R | A | H | H | $ | T | C | E | $ | R | Y | E | —NH2 | | 0 |
| - | | | | | | | | | | | | | | | | | | | | | | | | | | |
| Ac | | | | | | R | R | I | E | L | C | R | A | H | H | $ | T | C | E | $ | R | Y | E | —NH2 | | 1 |
| - | | | | | | | | | | | | | | | | | | | | | | | | | | |
| Ac | | | | | | R | A | I | E | L | C | R | R | H | H | $ | T | C | E | $ | R | Y | E | —NH2 | | 1 |
| - | | | | | | | | | | | | | | | | | | | | | | | | | | |
| Ac | | | | | | R | R | I | E | L | A | R | R | H | H | $ | T | C | E | $ | R | Y | E | —NH2 | | 2 |
| - | | | | | | | | | | | | | | | | | | | | | | | | | | |
| Ac | | | | | | R | R | I | E | L | C | R | R | H | H | $ | T | A | E | $ | R | Y | E | —NH2 | | 2 |
| - | | | | | | | | | | | | | | | | | | | | | | | | | | |
| Ac | | | | | | R | R | I | E | L | A | R | R | H | H | $ | T | A | E | $ | R | Y | E | —NH2 | | 2 |
| - | | | | | | | | | | | | | | | | | | | | | | | | | | |
| Ac | | | | | | R | R | I | $r8 | L | C | R | R | H | H | $ | T | C | E | A | R | Y | E | —NH2 | | 3 |
| - | | | | | | | | | | | | | | | | | | | | | | | | | | |
| Ac | | | | | | R | R | I | $r8 | L | A | R | R | H | H | $ | T | A | E | A | R | Y | E | —NH2 | | 3 |
| - | | | | | | | | | | | | | | | | | | | | | | | | | | |
| Ac | | E | R | L | R | R | R | I | E | L | C | R | R | H | H | $ | T | C | E | $ | R | Y | E | —NH2 | | 3 |
| - | | | | | | | | | | | | | | | | | | | | | | | | | | |
| Ac | | E | R | L | R | R | R | I | E | L | A | R | R | H | H | $ | T | A | E | $ | R | Y | E | —NH2 | | 3 |
| - | | | | | | | | | | | | | | | | | | | | | | | | | | |
| Ac | | E | R | L | R | R | R | I | $ | L | C | R | $ | H | H | S | T | C | E | A | R | Y | E | —NH2 | | 3 |
| - | | | | | | | | | | | | | | | | | | | | | | | | | | |
| Ac | | E | R | L | R | R | R | I | $ | L | A | R | $ | H | H | S | T | A | E | A | R | Y | E | —NH2 | | 3 |
| - | | | | | | | | | | | | | | | | | | | | | | | | | | |
| Ac | | E | R | L | R | R | R | I | E | L | C | R | $ | H | H | S | $ | C | E | A | R | Y | E | —NH2 | | 2 |
| - | | | | | | | | | | | | | | | | | | | | | | | | | | |
| Ac | | E | R | L | R | R | R | I | E | L | A | R | $ | H | H | S | $ | A | E | A | R | Y | E | —NH2 | | 2 |
| - | | | | | | | | | | | | | | | | | | | | | | | | | | |
| Ac | | E | R | L | R | R | $ | I | E | L | $ | R | R | H | H | S | T | C | E | A | R | Y | E | —NH2 | | 2 |
| - | | | | | | | | | | | | | | | | | | | | | | | | | | |
| Ac | | E | R | L | R | R | $ | I | E | L | $ | R | R | H | H | S | T | A | E | A | R | Y | E | —NH2 | | 2 |
| - | | | | | | | | | | | | | | | | | | | | | | | | | | |
| Ac | | E | R | L | R | R | R | I | $r8 | L | C | R | R | H | H | $ | T | C | E | A | R | Y | E | —NH2 | | 4 |
| - | | | | | | | | | | | | | | | | | | | | | | | | | | |
| Ac | | E | R | L | R | R | R | I | $r8 | L | A | R | R | II | H | $ | T | A | E | A | R | Y | E | —NH2 | | 4 |
| - | | | | | | | | | | | | | | | | | | | | | | | | | | |
| Ac | | E | R | L | R | R | A | I | $r8 | L | A | R | A | H | H | $ | T | A | E | A | R | Y | E | —NH2 | | 2 |
| - | | | | | | | | | | | | | | | | | | | | | | | | | | |

### Peptidomimetic Macrocycles of the Invention

In some embodiments, a peptidomimetic macrocycle of the invention has the Formula (I):

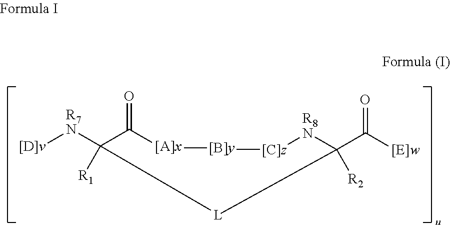

wherein:

each A, C, D, and E is independently a natural or non-natural amino acid;

B is a natural or non-natural amino acid, amino acid analog,

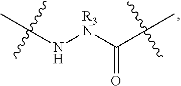

### [—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula -L₁-L₂-;

L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

v and w are independently integers from 1-1000;

u, x, y and z are independently integers from 0-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges. Similarly, when u is greater than 1, each compound of the invention may encompass peptidomimetic macrocycles which are the same or different. For example, a compound of the invention may comprise peptidomimetic macrocycles comprising different linker lengths or chemical compositions.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

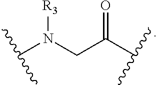

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In one embodiment, the peptidomimetic macrocycle of Formula (I) is:

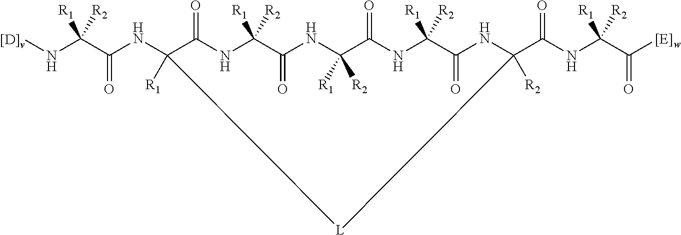

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle of Formula (I) is:

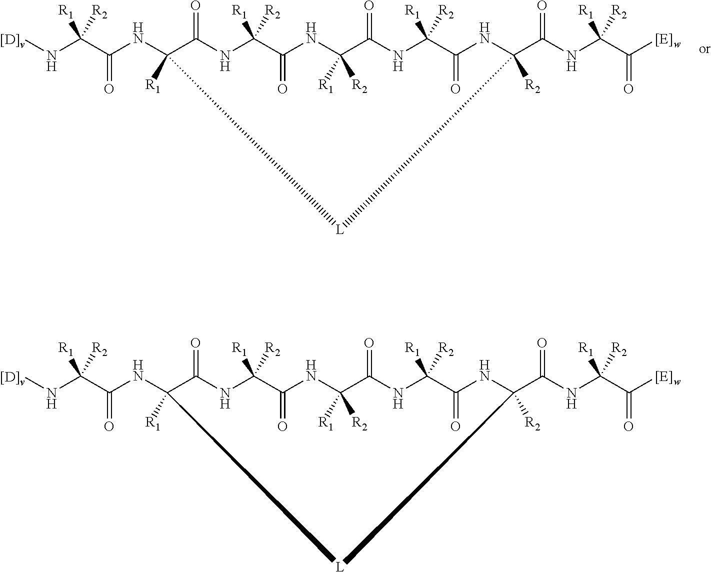

In other embodiments, the peptidomimetic macrocycle of Formula (I) is a compound of any of the formulas shown below:

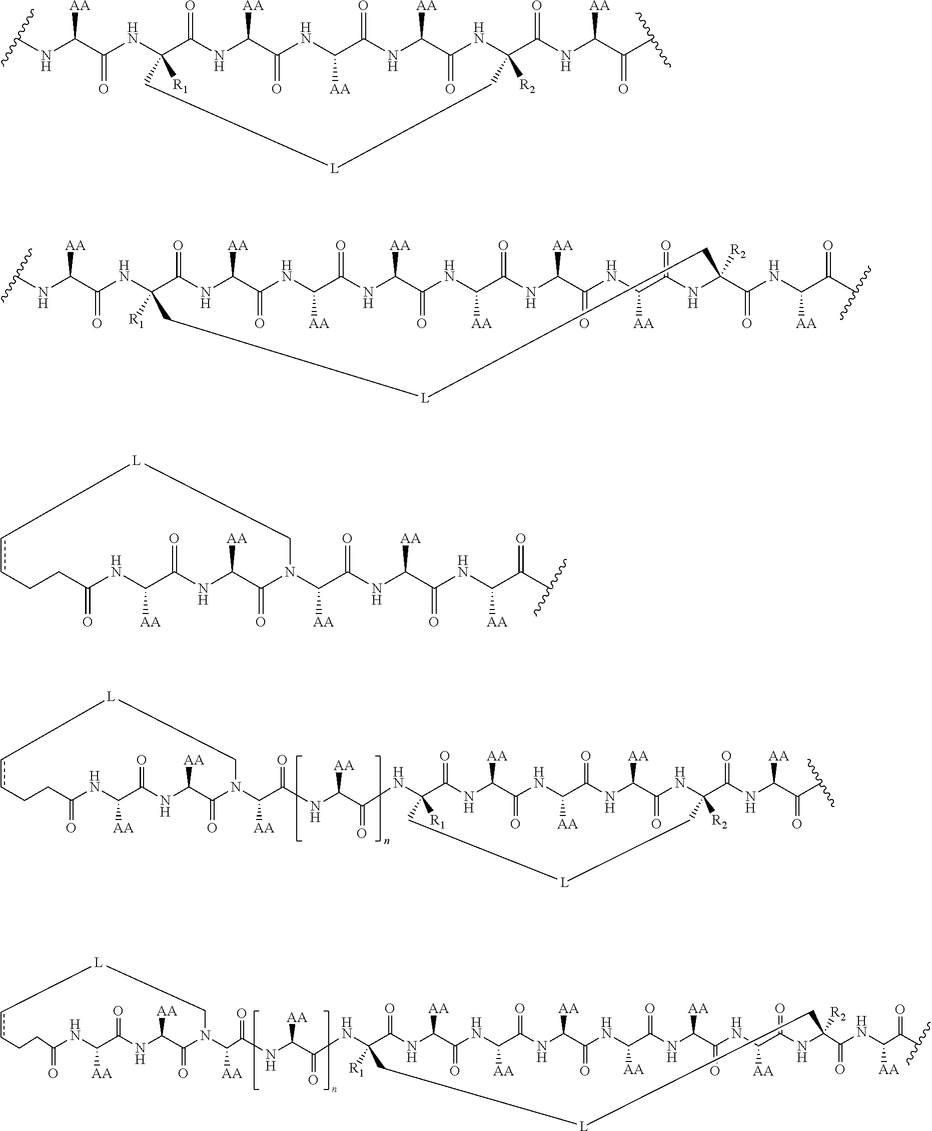

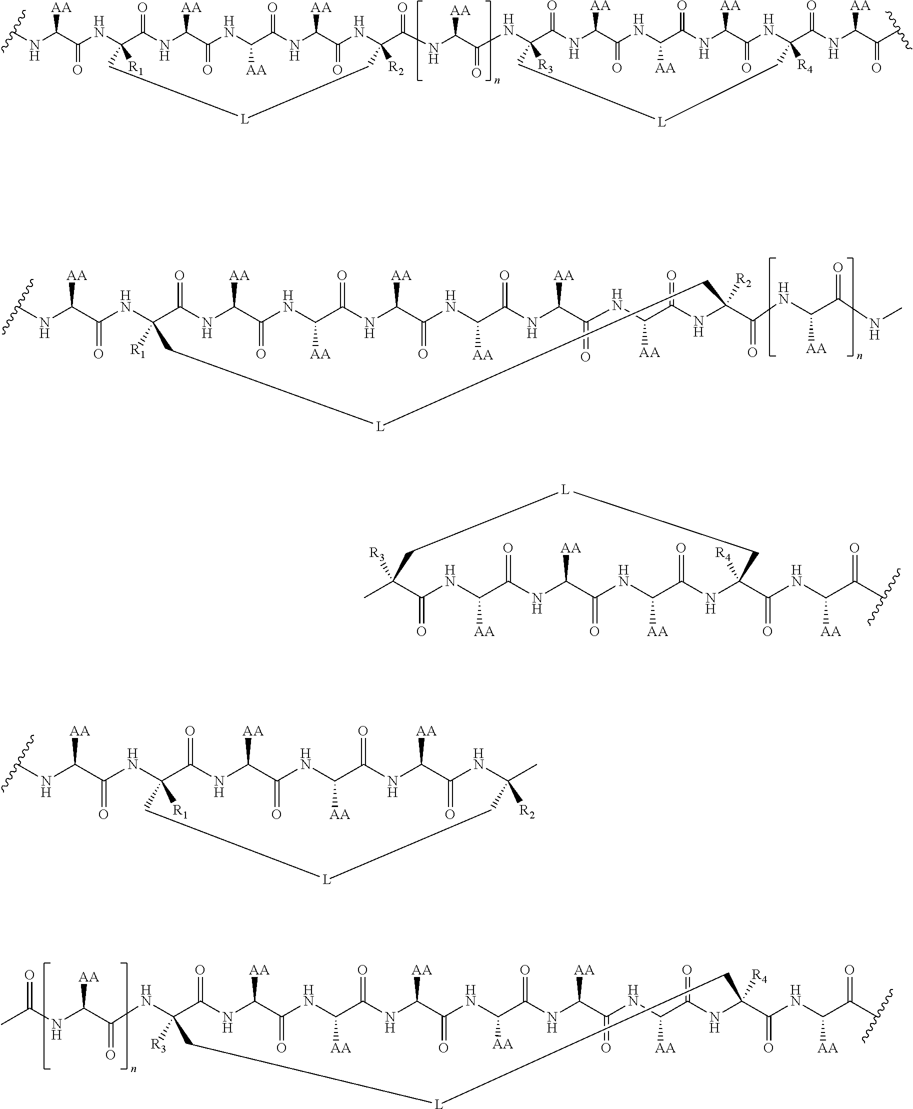

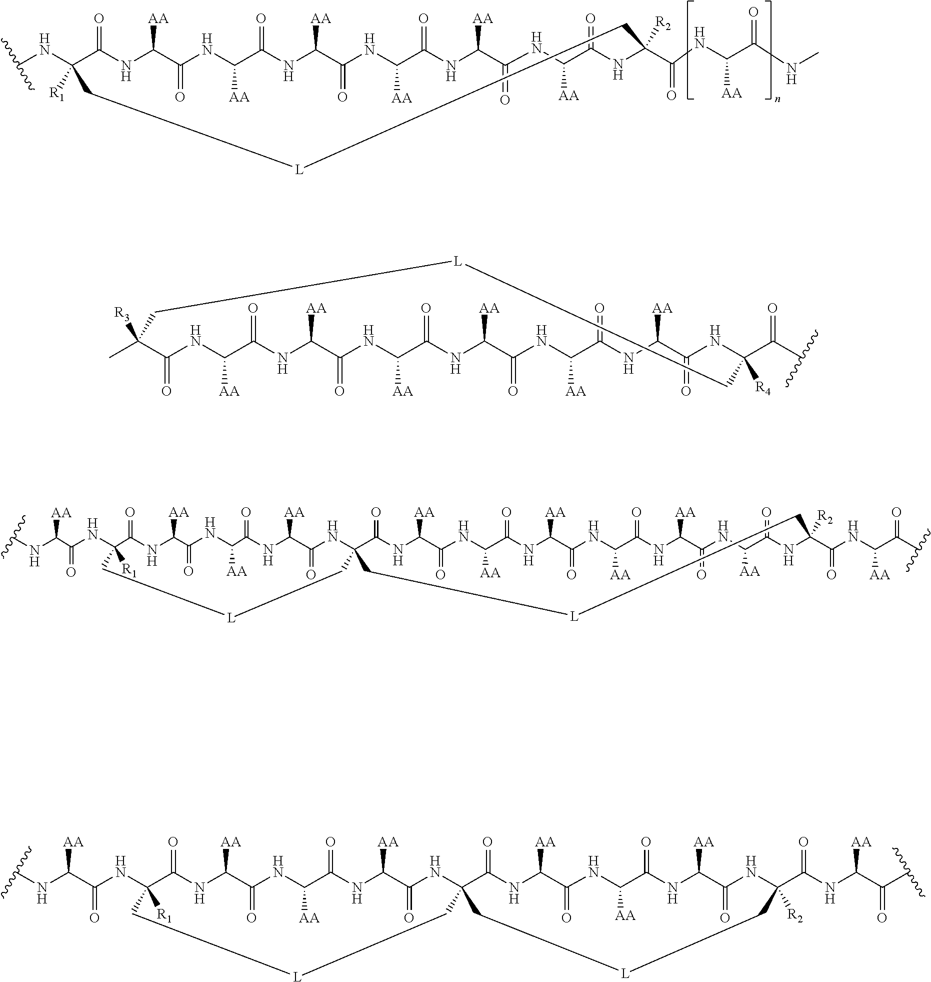

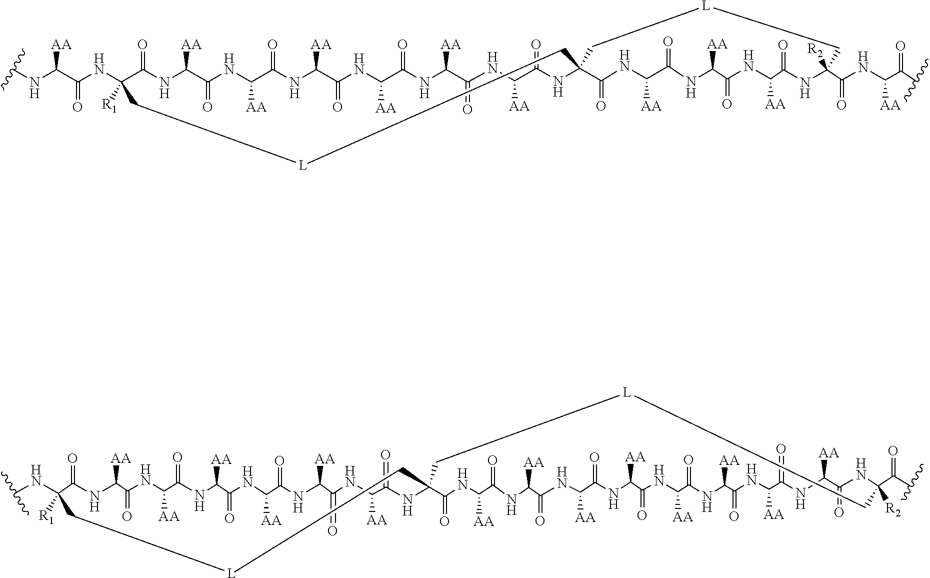

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, n is 0. In other embodiments, n is less than 50.

Exemplary embodiments of the macrocycle-forming linker L are shown below.

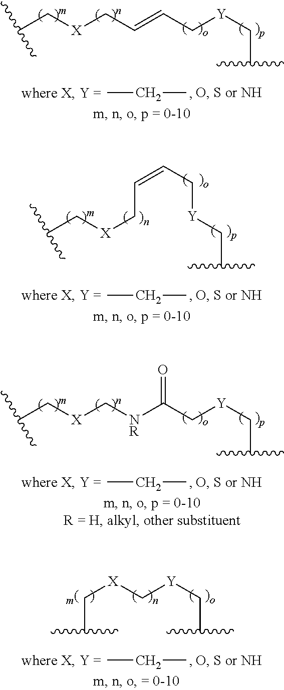

In some embodiments, the peptidomimetic macrocycles of the invention have the Formula (II):

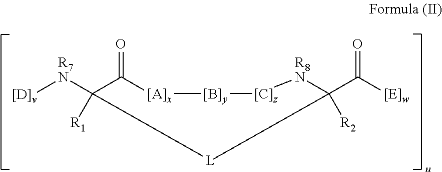

wherein:

each A, C, D, and E is independently a natural or non-natural amino acid;

B is a natural or non-natural amino acid, amino acid analog

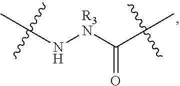

### [—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula

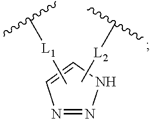

L₁, L₂ and L₃ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

v and w are independently integers from 1-1000;

u, x, y and z are independently integers from 0-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

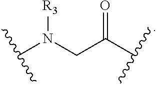

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker L are shown below.

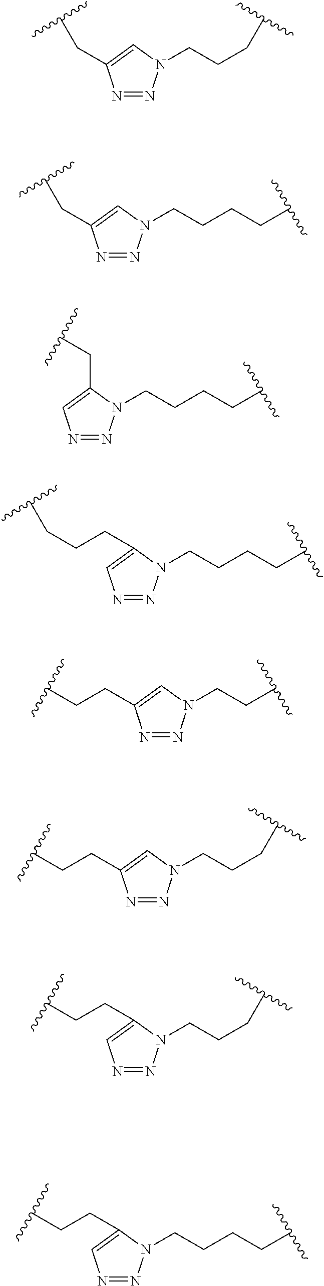

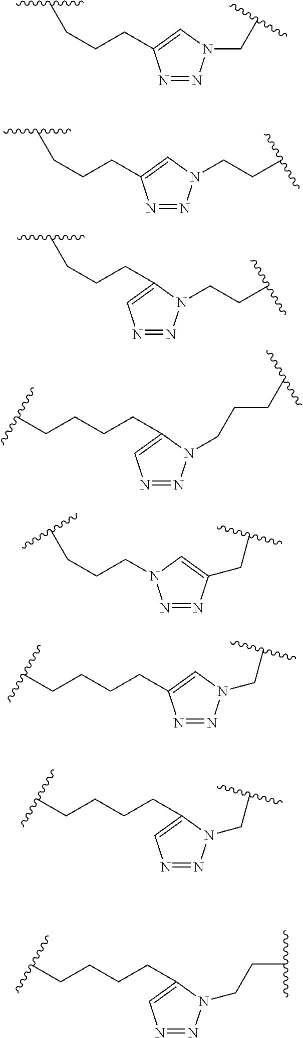

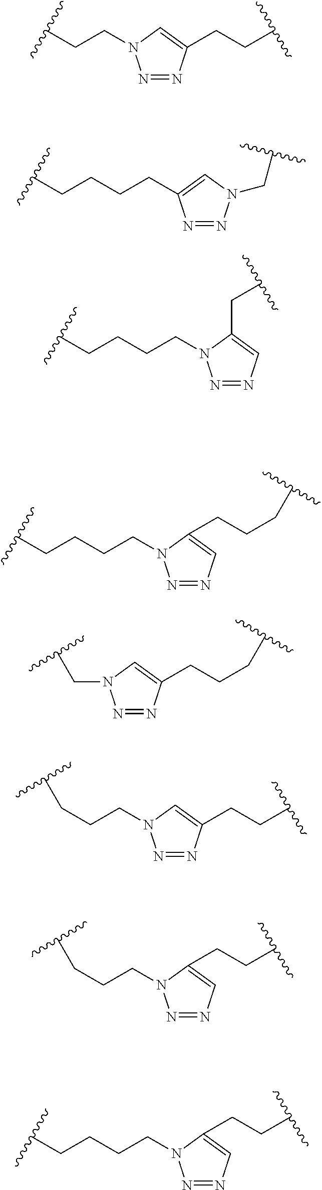

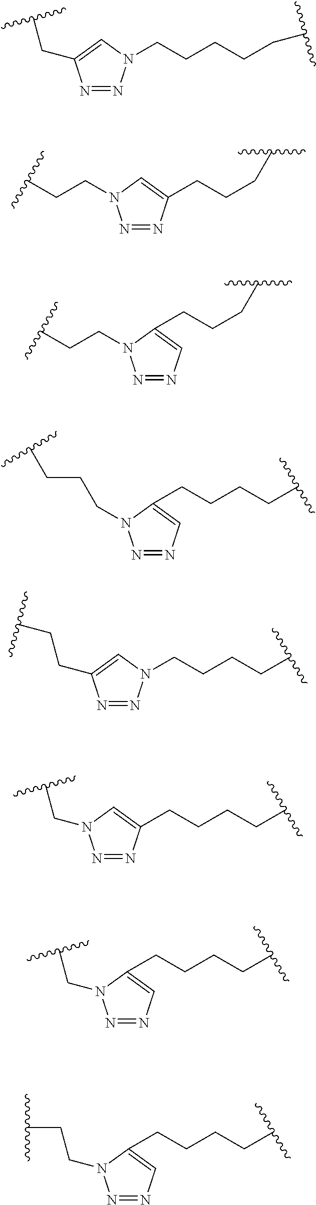

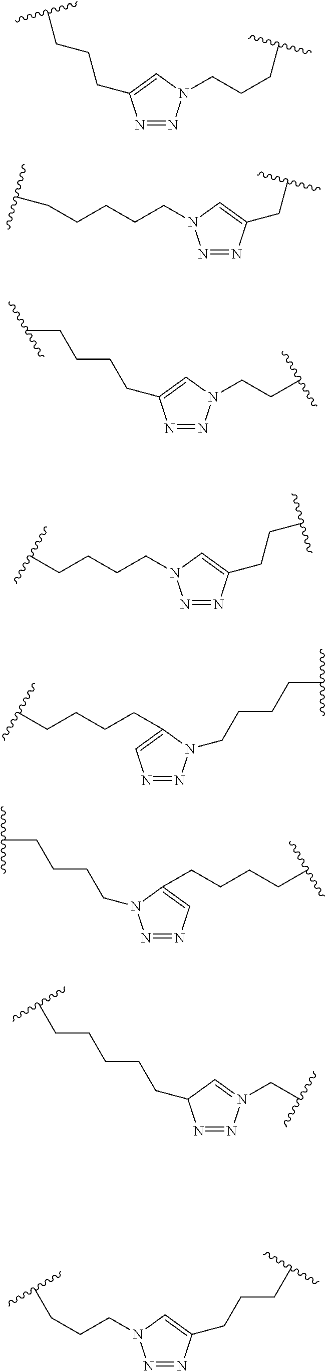

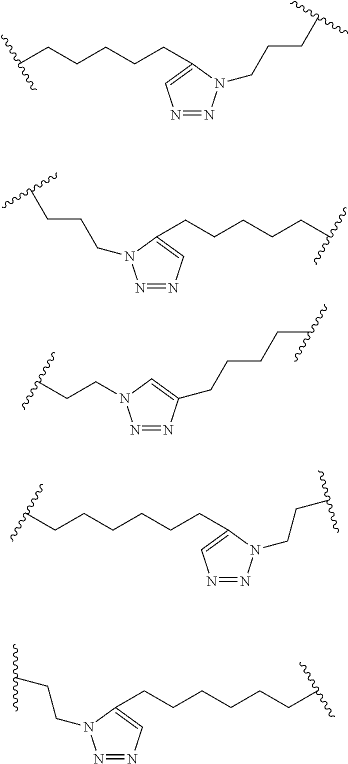

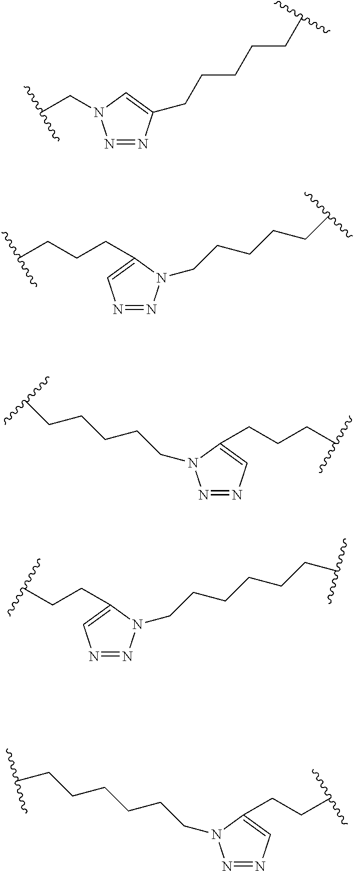

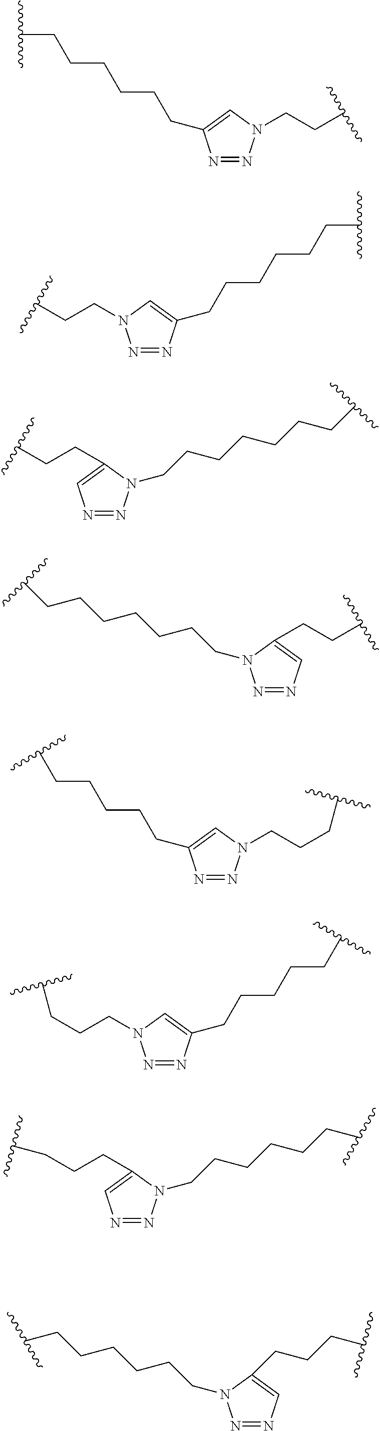

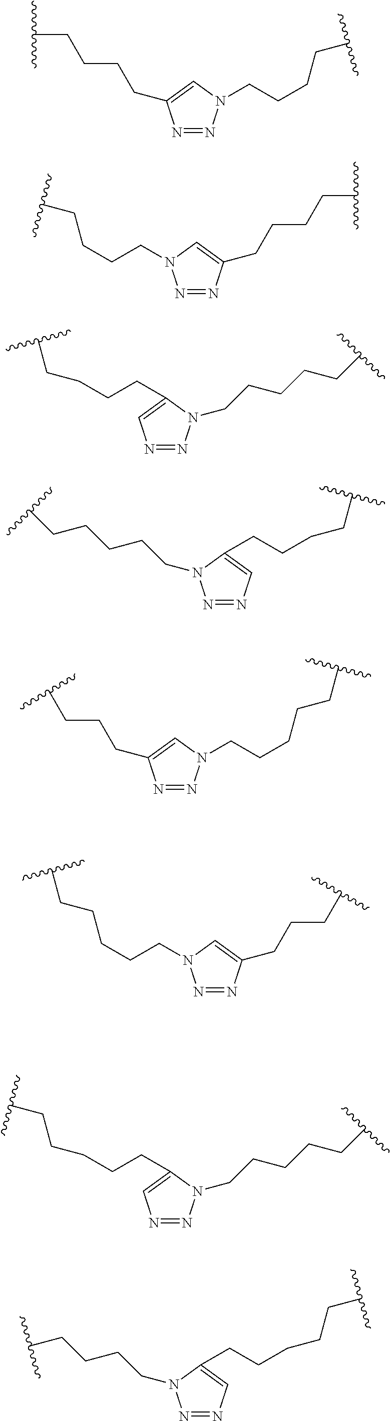

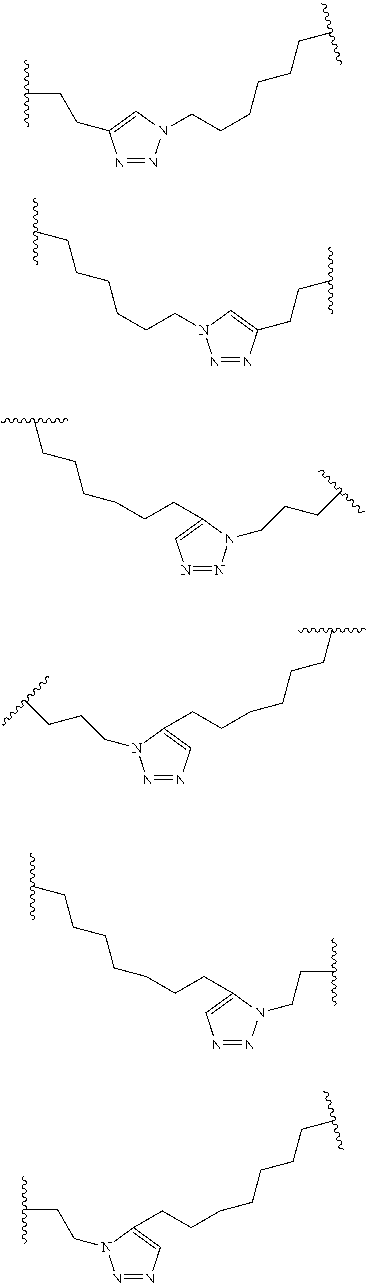

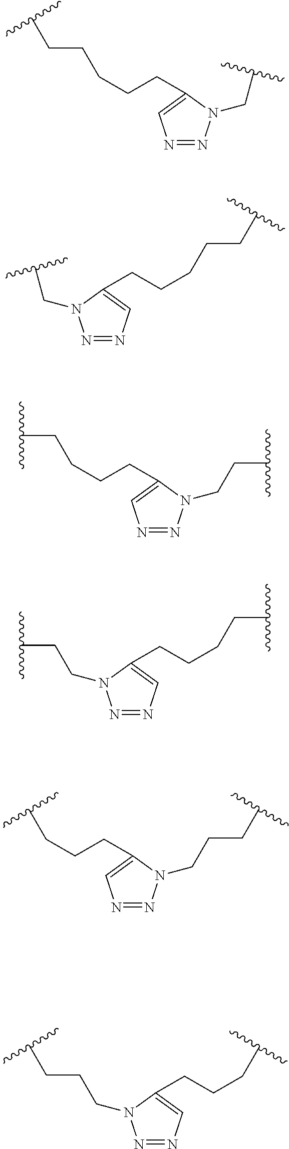

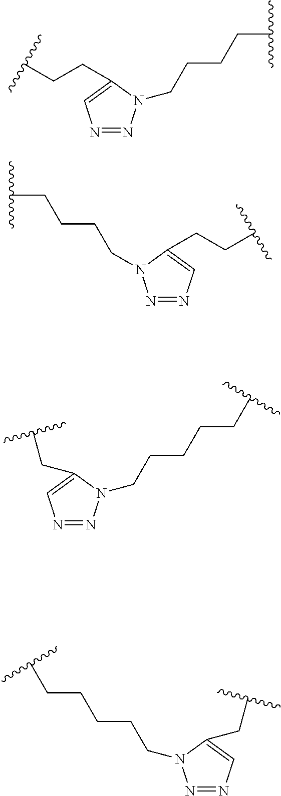

In other embodiments, the invention provides peptidomimetic macrocycles of Formula (III):

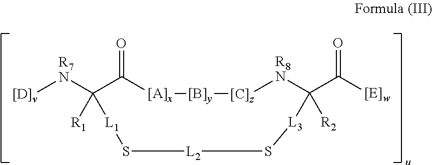

wherein:
each A, C, D, and E is independently a natural or non-natural amino acid;
B is a natural or non-natural amino acid, amino acid analog,

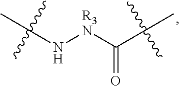

### [—NH-L₄-CO—], [—NH-L₄-SO₂—], or [—NH-L₄-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, unsubstituted or substituted with R₅;
L₁, L₂, L₃ and L₄ are independently alkylene, alkenylene, alkynylene, heteroalkylene, cycloalkylene, heterocycloalkylene, cycloarylene, heterocycloarylene or [—R₄—K—R₄—]ₙ, each being unsubstituted or substituted with R₅;

### K is O, S, SO, SO₂, CO, CO₂, or CONR₃;

each R₄ is alkylene, alkenylene, alkynylene, heteroalkylene, cycloalkylene, heterocycloalkylene, arylene, or heteroarylene;
each R₅ is independently halogen, alkyl, —OR₆, —N(R₆)₂, —SR₆, —SOR₆, SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, unsubstituted or substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, unsubstituted or substituted with R₅, or part of a cyclic structure with an E residue;
v and w are independently integers from 1-1000;
u, x, y and z are independently integers from 0-10; and
n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

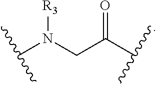

In other embodiments, the length of the macrocycle-forming linker [-L₁-S-L₂-S-L₃-] as measured from a first Cα to a second Cα is selected to stabilize a desired secondary peptide structure, such as an α-helix formed by residues of the peptidomimetic macrocycle including, but not necessarily limited to, those between the first Cα to a second Cα.

Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985. In some embodiments, the thiol moieties are the side chains of the amino acid residues L-cysteine, D-cysteine, α-methyl-L cysteine, α-methyl-D-cysteine, L-homocysteine, D-homocysteine, α-methyl-L-homocysteine or α-methyl-D-homocysteine. A bis-alkylating reagent is of the general formula X-L₂-Y wherein L₂ is a linker moiety and X and Y are leaving groups that are displaced by —SH moieties to form bonds with L₂. In some embodiments, X and Y are halogens such as I, Br, or Cl.

In other embodiments, D and/or E in the compound of Formula I, II or III are further modified in order to facilitate cellular uptake. In some embodiments, lipidating or PEGylating a peptidomimetic macrocycle facilitates cellular uptake, increases bioavailability, increases blood circulation, alters pharmacokinetics, decreases immunogenicity and/or decreases the needed frequency of administration.

In other embodiments, at least one of [D] and [E] in the compound of Formula I, II or III represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers.

In the peptidomimetic macrocycles of the invention, any of the macrocycle-forming linkers described herein may be used in any combination with any of the sequences shown in Tables 1-4 and also with any of the R-substituents indicated herein.

In some embodiments, the peptidomimetic macrocycle comprises at least one α-helix motif. For example, A, B and/or C in the compound of Formula I, II or III include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix. In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix. Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds. Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms. Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In other embodiments, the invention provides peptidomimetic macrocycles of Formula (IV) or (IVa):

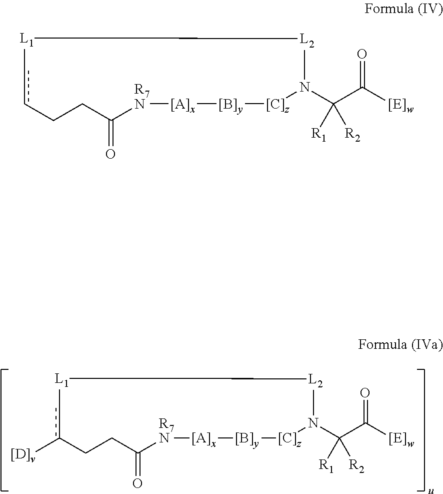

wherein:

each A, C, D, and E is independently a natural or non-natural amino acid;

B is a natural or non-natural amino acid, amino acid analog,

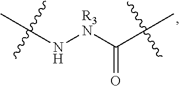

### [—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula -L₁-L₂-;

L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR_{B}, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅;

v and w are independently integers from 1-1000;

u, x, y and z are independently integers from 0-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 1. In some embodiments of the invention, x+y+z is at least 2. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

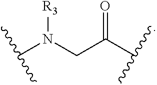

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker -L₁-L₂- are shown below.

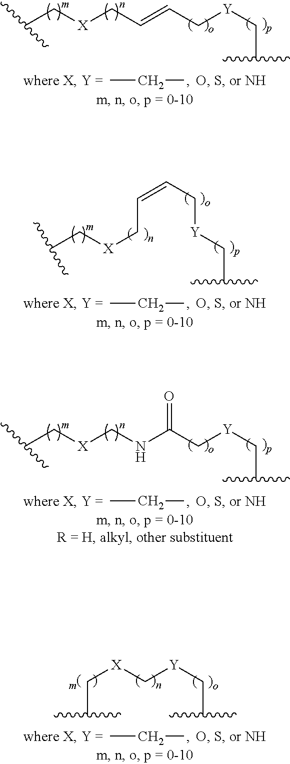

### Preparation of Peptidomimetic Macrocycles

Peptidomimetic macrocycles of the invention may be prepared by any of a variety of methods known in the art. For example, any of the residues indicated by “X” in Tables 1, 2, 3 or 4 may be substituted with a residue capable of forming a crosslinker with a second residue in the same molecule or a precursor of such a residue.

Various methods to effect formation of peptidomimetic macrocycles are known in the art. For example, the preparation of peptidomimetic macrocycles of Formula I is described in Schafineister et al., J. Am. Chem. Soc. 122:5891-5892 (2000); Schafmeister & Verdin, J. Am. Chem. Soc. 122:5891 (2005); Walensky et al., Science 305:1466-1470 (2004); and U.S. Pat. No. 7,192,713. The α,α-disubstituted amino acids and amino acid precursors disclosed in the cited references may be employed in synthesis of the peptidomimetic macrocycle precursor polypeptides. For example, the “S5-olefin amino acid” is (S)-α-(2′-pentenyl) alanine and the “R8 olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle.

In other embodiments, the peptidomimetic macrocyles of the invention are of Formula IV or IVa. Methods for the preparation of such macrocycles are described, for example, in U.S. Pat. No. 7,202,332.

In some embodiments, the synthesis of these peptidomimetic macrocycles involves a multi-step process that features the synthesis of a peptidomimetic precursor containing an azide moiety and an alkyne moiety; followed by contacting the peptidomimetic precursor with a macrocyclization reagent to generate a triazole-linked peptidomimetic macrocycle. Such a process is described, for example, in U.S. application Ser. No. 12/037,041, filed on Feb. 25, 2008. Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985.

In some embodiments, an azide is linked to the α-carbon of a residue and an alkyne is attached to the α-carbon of another residue. In some embodiments, the azide moieties are azido-analogs of amino acids L-lysine, D-lysine, alpha-methyl-L-lysine, alpha-methyl-D-lysine, L-ornithine, D-ornithine, alpha-methyl-L-ornithine or alpha-methyl-D-ornithine. In another embodiment, the alkyne moiety is L-propargylglycine. In yet other embodiments, the alkyne moiety is an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid and (R)-2-amino-2-methyl-8-nonynoic acid.

In some embodiments, the invention provides a method for synthesizing a peptidomimetic macrocycle, the method comprising the steps of contacting a peptidomimetic precursor of Formula V or Formula VI:

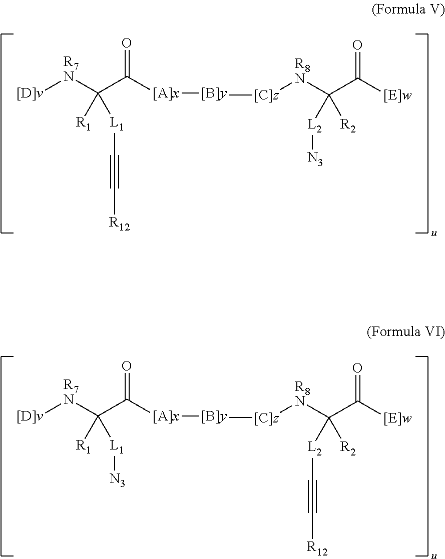

with a macrocyclization reagent;

wherein v, w, x, y, z, A, B, C, D, E, R₁, R₂, R₇, R₈, L₁ and L₂ are as defined for Formula (II); R₁₂ is —H when the macrocyclization reagent is a Cu reagent and R₁₂ is —H or alkyl when the macrocyclization reagent is a Ru reagent; and further wherein said contacting step results in a covalent linkage being formed between the alkyne and azide moiety in Formula III or Formula IV. For example, R₁₂ may be methyl when the macrocyclization reagent is a Ru reagent.

In the peptidomimetic macrocycles of the invention, at least one of R₁ and R₂ is alkyl, alkenyl, alkynyl, arylalkyl, cycloalkyl, cycloalkylalkyl, heteroalkyl, or heterocycloalkyl, unsubstituted or substituted with halo-. In some embodiments, both R₁ and R₂ are independently alkyl, alkenyl, alkynyl, arylalkyl, cycloalkyl, cycloalkylalkyl, heteroalkyl, or heterocycloalkyl, unsubstituted or substituted with halo-. In some embodiments, at least one of A, B, C, D or E is an α,α-disubstituted amino acid. In one example, B is an α,α-disubstituted amino acid. For instance, at least one of A, B, C, D or E is 2-aminoisobutyric acid.

For example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl. The macrocyclization reagent may be a Cu reagent or a Ru reagent.

In some embodiments, the peptidomimetic precursor is purified prior to the contacting step. In other embodiments, the peptidomimetic macrocycle is purified after the contacting step. In still other embodiments, the peptidomimetic macrocycle is refolded after the contacting step. The method may be performed in solution, or, alternatively, the method may be performed on a solid support.

Also envisioned herein is performing the method of the invention in the presence of a target macromolecule that binds to the peptidomimetic precursor or peptidomimetic macrocycle under conditions that favor said binding. In some embodiments, the method is performed in the presence of a target macromolecule that binds preferentially to the peptidomimetic precursor or peptidomimetic macrocycle under conditions that favor said binding. The method may also be applied to synthesize a library of peptidomimetic macrocycles.

In some embodiments, the alkyne moiety of the peptidomimetic precursor of Formula V or Formula VI is a sidechain of an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid, and (R)-2-amino-2-methyl-8-nonynoic acid. In other embodiments, the azide moiety of the peptidomimetic precursor of Formula V or Formula VI is a sidechain of an amino acid selected from the group consisting of ε-azido-L-lysine, ε-azido-D-lysine, ε-azido-α-methyl-L-lysine, ε-azido-α-methyl-D-lysine, δ-azido-α-methyl-L-ornithine, and δ-azido-α-methyl-D-ornithine.

In some embodiments, x+y+z is 3, and A, B and C are independently natural or non-natural amino acids. In other embodiments, x+y+z is 6, and A, B and C are independently natural or non-natural amino acids.

In some embodiments, the contacting step is performed in a solvent selected from the group consisting of protic solvent, aqueous solvent, organic solvent, and mixtures thereof. For example, the solvent may be chosen from the group consisting of H₂O, THF, THF/H₂O, tBuOH/H₂O, DMF, DIPEA, CH₃CN or CH₂Cl₂, ClCH₂CH₂Cl or a mixture thereof. The solvent may be a solvent which favors helix formation.

Alternative but equivalent protecting groups, leaving groups or reagents are substituted, and certain of the synthetic steps are performed in alternative sequences or orders to produce the desired compounds. Synthetic chemistry transformations and protecting group methodologies (protection and deprotection) useful in synthesizing the compounds described herein include, for example, those such as described in Larock, Comprehensive Organic Transformations, VCH Publishers (1989); Greene and Wuts, Protective Groups in Organic Synthesis, 2d. Ed., John Wiley and Sons (1991); Fieser and Fieser, Fieser and Fieser's Reagents for Organic Synthesis, John Wiley and Sons (1994); and Paquette, ed., Encyclopedia of Reagents for Organic Synthesis, John Wiley and Sons (1995), and subsequent editions thereof.

The peptidomimetic macrocycles of the invention are made, for example, by chemical synthesis methods, such as described in Fields et al., Chapter 3 in Synthetic Peptides: A User's Guide, ed. Grant, W. H. Freeman & Co., New York, N.Y., 1992, p. 77. Hence, for example, peptides are synthesized using the automated Merrifield techniques of solid phase synthesis with the amine protected by either tBoc or Fmoc chemistry using side chain protected amino acids on, for example, an automated peptide synthesizer (e.g., Applied Biosystems (Foster City, Calif.), Model 430A, 431, or 433).

One manner of producing the peptidomimetic precursors and peptidomimetic macrocycles described herein uses solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Side chain functional groups are protected as necessary with base stable, acid labile groups.

Longer peptidomimetic precursors are produced, for example, by conjoining individual synthetic peptides using native chemical ligation. Alternatively, the longer synthetic peptides are biosynthesized by well known recombinant DNA and protein expression techniques. Such techniques are provided in well-known standard manuals with detailed protocols. To construct a gene encoding a peptidomimetic precursor of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptidomimetic precursors are made, for example, in a high-throughput, combinatorial fashion using, for example, a high-throughput polychannel combinatorial synthesizer (e.g., Thuramed TETRAS multichannel peptide synthesizer from CreoSalus, Louisville, Ky. or Model Apex 396 multichannel peptide synthesizer from AAPPTEC, Inc., Louisville, Ky.).

The following synthetic schemes are provided solely to illustrate the present invention and are not intended to limit the scope of the invention, as described herein. To simplify the drawings, the illustrative schemes depict azido amino acid analogs ε-azido-α-methyl-L-lysine and ε-azido-α-methyl-D-lysine, and alkyne amino acid analogs L-propargylglycine, (S)-2-amino-2-methyl-4-pentynoic acid, and (S)-2-amino-2-methyl-6-heptynoic acid. Thus, in the following synthetic schemes, each R₁, R₂, R₇ and R₈ is —H; each L₁ is —(CH₂)₄—; and each L₂ is —(CH₂)—. However, as noted throughout the detailed description above, many other amino acid analogs can be employed in which R₁, R₂, R₇, R₈, L₁ and L₂ can be independently selected from the various structures disclosed herein.

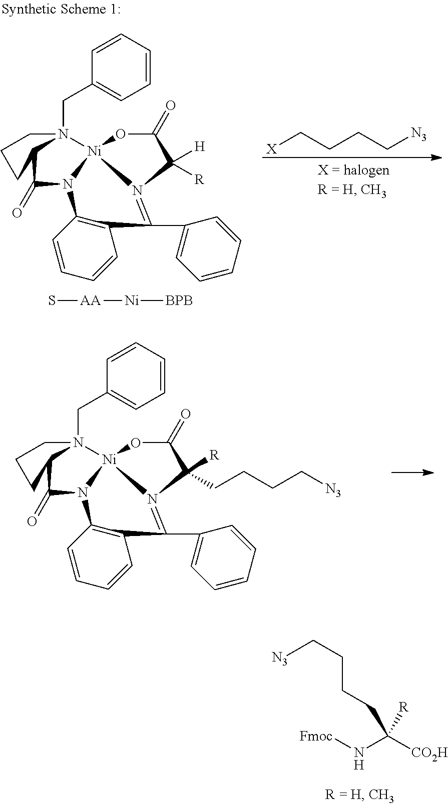

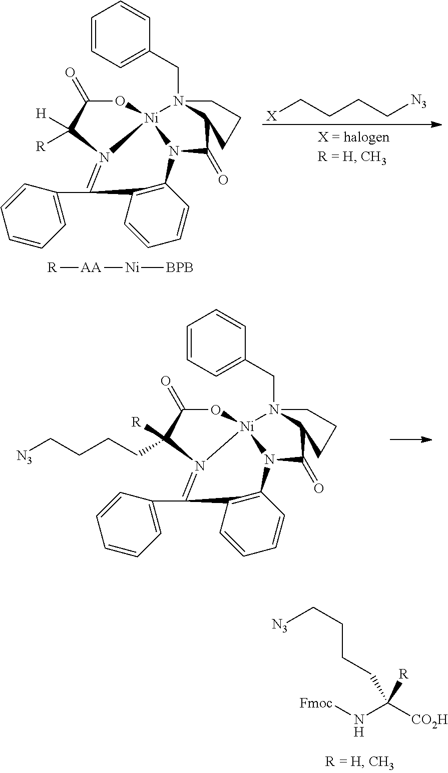

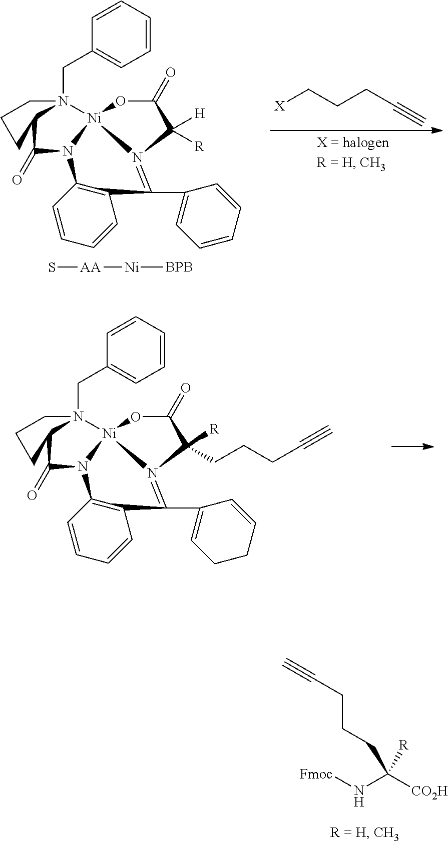

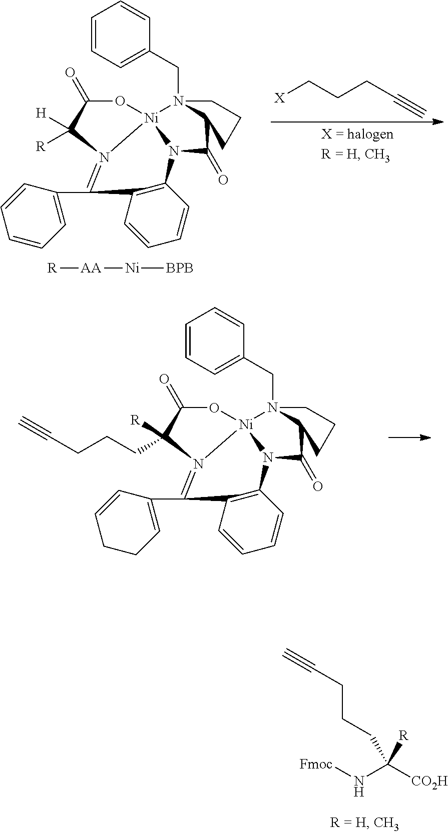

Synthetic Scheme 1 describes the preparation of several compounds of the invention. Ni(II) complexes of Schiff bases derived from the chiral auxiliary (S)-2-[N—(N′-benzylprolyl)amino]benzophenone (BPB) and amino acids such as glycine or alanine are prepared as described in Belokon et al. (1998), Tetrahedron Asymm. 9:4249-4252. The resulting complexes are subsequently reacted with alkylating reagents comprising an azido or alkynyl moiety to yield enantiomerically enriched compounds of the invention. If desired, the resulting compounds can be protected for use in peptide synthesis.

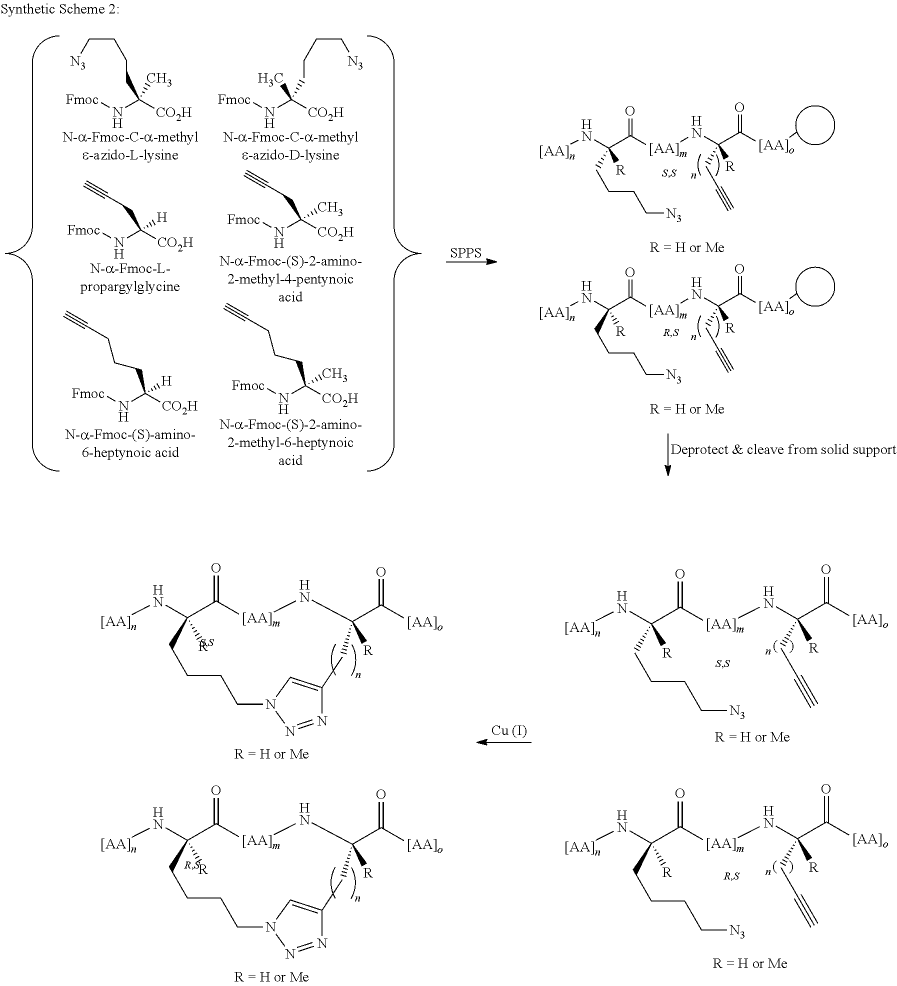

In the general method for the synthesis of peptidomimetic macrocycles shown in Synthetic Scheme 2, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Cu(I) in organic or aqueous solutions (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). In one embodiment, the triazole forming reaction is performed under conditions that favor α-helix formation. In one embodiment, the macrocyclization step is performed in a solvent chosen from the group consisting of H₂O, THF, CH₃CN, DMF, DIPEA, tBuOH or a mixture thereof. In another embodiment, the macrocyclization step is performed in DMF. In some embodiments, the macrocyclization step is performed in a buffered aqueous or partially aqueous solvent.

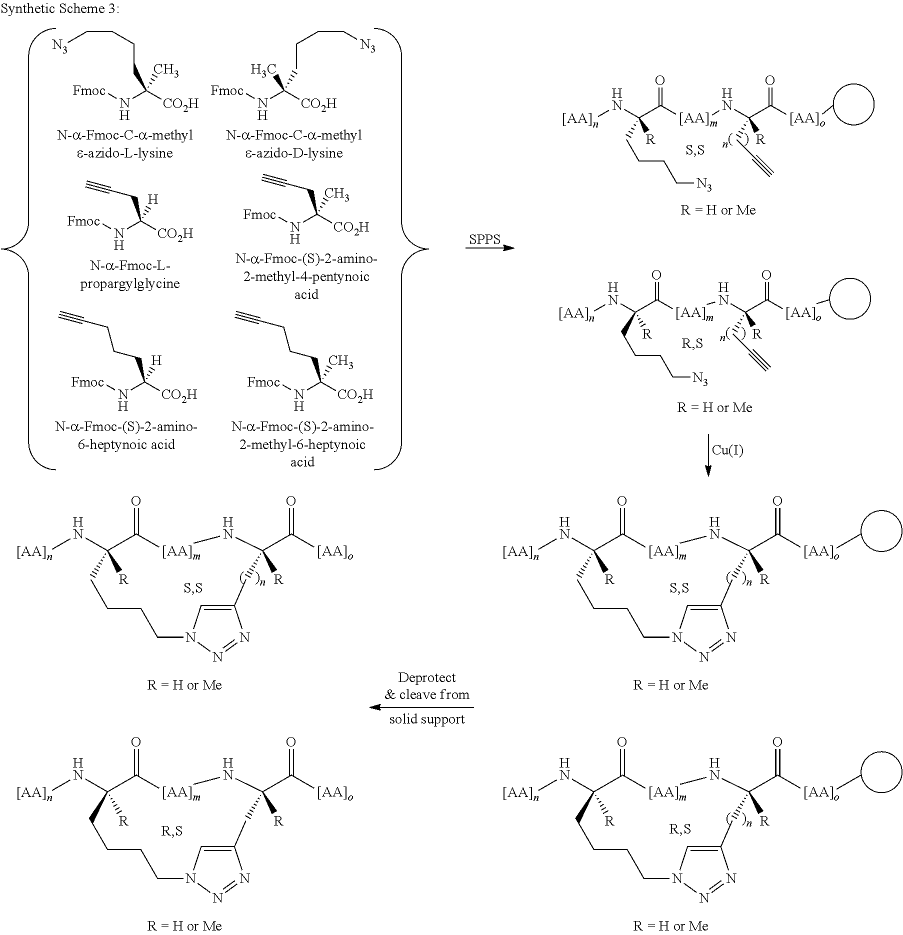

In the general method for the synthesis of peptidomimetic macrocycles shown in Synthetic Scheme 3, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is reacted with a macrocyclization reagent such as a Cu(I) reagent on the resin as a crude mixture (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). The resultant triazole-containing peptidomimetic macrocycle is then deprotected and cleaved from the solid-phase resin by standard conditions (e.g., strong acid such as 95% TFA). In some embodiments, the macrocyclization step is performed in a solvent chosen from the group consisting of CH₂Cl₂, ClCH₂CH₂Cl, DMF, THF, NMP, DIPEA, 2,6-lutidine, pyridine, DMSO, H₂O or a mixture thereof. In some embodiments, the macrocyclization step is performed in a buffered aqueous or partially aqueous solvent.

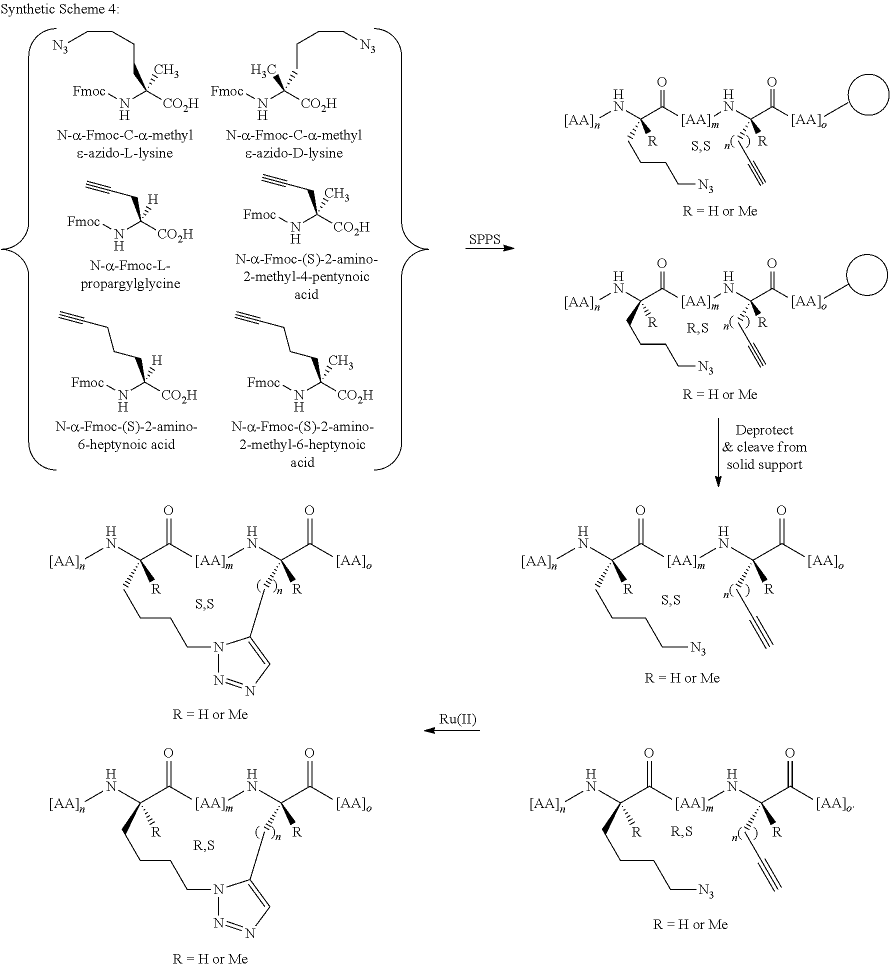

In the general method for the synthesis of peptidomimetic macrocycles shown in Synthetic Scheme 4, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Ru(II) reagents, for example Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of DMF, CH₃CN and THF.

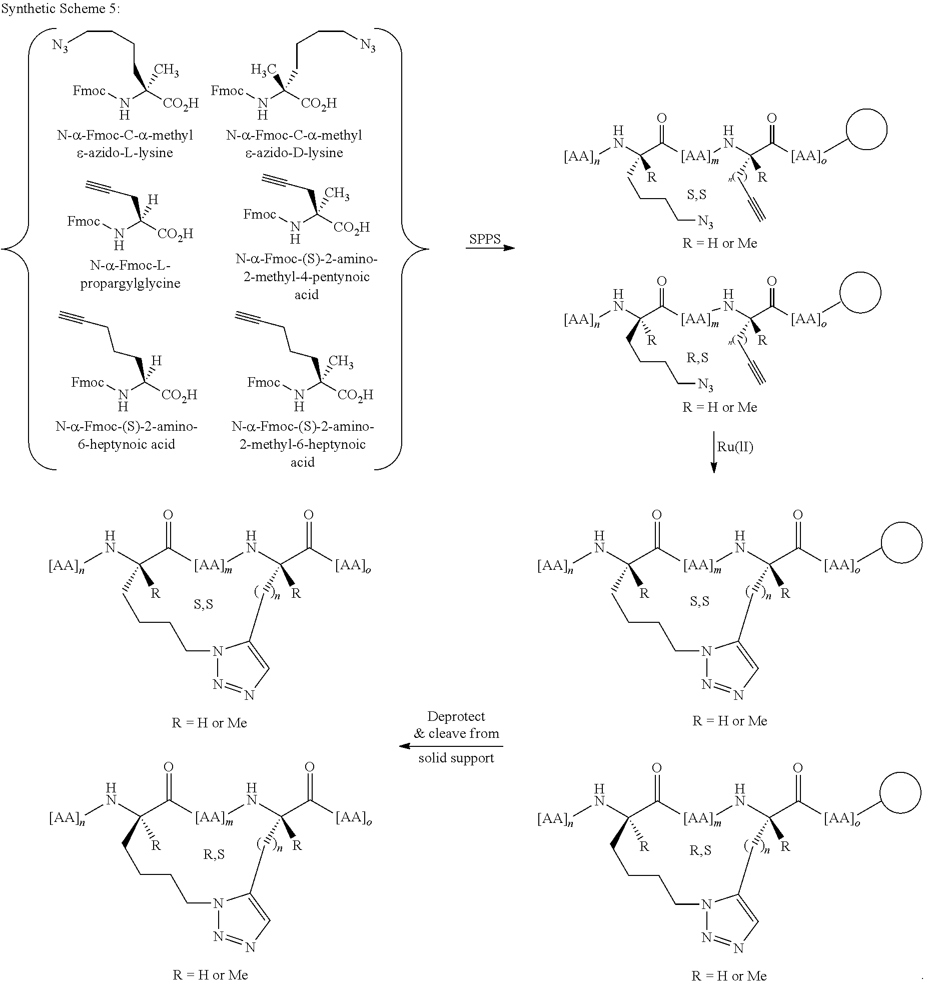

In the general method for the synthesis of peptidomimetic macrocycles shown in Synthetic Scheme 5, the peptidomimetic precursor contains an azide moiety and an alkyne moiety and is synthesized by solid-phase peptide synthesis (SPPS) using the commercially available amino acidN-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is reacted with a macrocyclization reagent such as a Ru(II) reagent on the resin as a crude mixture. For example, the reagent can be Cp*RuCl(PPh₃)₂ or [Cp*RuCl]₄ (Rasmussen et al, (2007), Org. Lett. 9:5337-5339; Zhang et al. (2005), J. Am. Chem. Soc. 127:15998-15999). In some embodiments, the macrocyclization step is performed in a solvent chosen from the group consisting of CH₂Cl₂, ClCH₂CH₂Cl, CH₃CN, DMF, and THF.

The present invention contemplates the use of non-naturally-occurring amino acids and amino acid analogs in the synthesis of the peptidomimetic macrocycles described herein. Any amino acid or amino acid analog amenable to the synthetic methods employed for the synthesis of stable triazole containing peptidomimetic macrocycles can be used in the present invention. For example, L-propargylglycine is contemplated as a useful amino acid in the present invention. However, other alkyne-containing amino acids that contain a different amino acid side chain are also useful in the invention. For example, L-propargylglycine contains one methylene unit between the α-carbon of the amino acid and the alkyne of the amino acid side chain. The invention also contemplates the use of amino acids with multiple methylene units between the α-carbon and the alkyne. Also, the azido-analogs of amino acids L-lysine, D-lysine, alpha-methyl-L-lysine, and alpha-methyl-D-lysine are contemplated as useful amino acids in the present invention. However, other terminal azide amino acids that contain a different amino acid side chain are also useful in the invention. For example, the azido-analog of L-lysine contains four methylene units between the α-carbon of the amino acid and the terminal azide of the amino acid side chain. The invention also contemplates the use of amino acids with fewer than or greater than four methylene units between the α-carbon and the terminal azide. Table 2 shows some amino acids useful in the preparation of peptidomimetic macrocycles of the invention.

TABLE 2

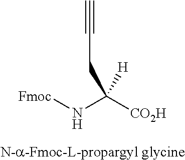

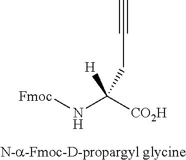

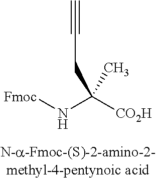

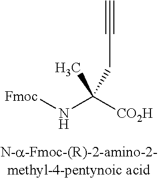

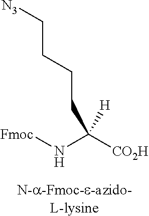

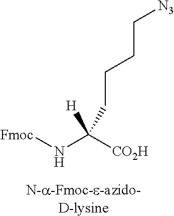

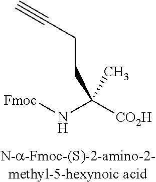

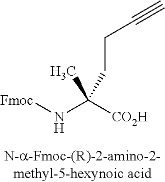

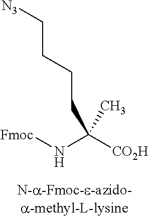

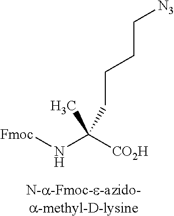

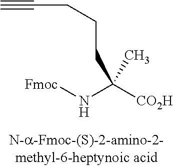

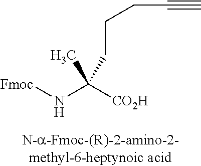

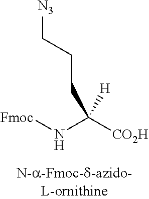

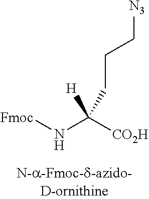

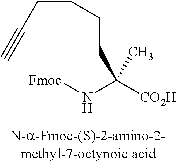

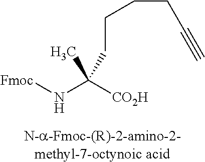

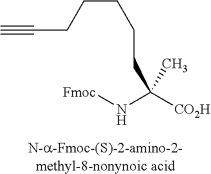

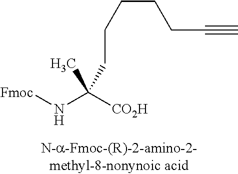

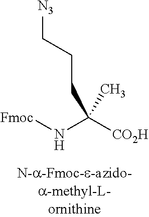

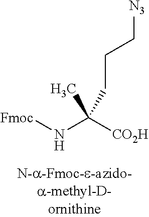

Table 2 shows exemplary amino acids useful in the preparation of peptidomimetic macrocycles of the invention.

In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-propargylglycine, α-methyl-D-propargylglycine, ε-azido-alpha-methyl-L-lysine, and ε-azido-alpha-methyl-D-lysine. In some embodiments the amino acid analogs are N-alkylated, e.g., N-methyl-L-propargylglycine, N-methyl-D-propargylglycine, N-methyl-ε-azido-L-lysine, and N-methyl-ε-azido-D-lysine.

In some embodiments, the —NH moiety of the amino acid is protected using a protecting group, including without limitation -Fmoc and -Doc. In other embodiments, the amino acid is not protected prior to synthesis of the peptidomimetic macrocycle.

In other embodiments, peptidomimetic macrocycles of Formula III are synthesized. The preparation of such macrocycles is described, for example, in U.S. application Ser. No. 11/957,325, filed on Dec. 17, 2007. The following synthetic schemes describe the preparation of such compounds. To simplify the drawings, the illustrative schemes depict amino acid analogs derived from L- or D-cysteine, in which L₁ and L₃ are both —(CH₂)—. However, as noted throughout the detailed description above, many other amino acid analogs can be employed in which L₁ and L₃ can be independently selected from the various structures disclosed herein. The symbols “[AA]ₘ”, “[AA]ₙ,”, “[AA]ₒ,” represent a sequence of amide bond-linked moieties such as natural or unnatural amino acids. As described previously, each occurrence of “AA” is independent of any other occurrence of “AA”, and a formula such as “[AA]ₘ” encompasses, for example, sequences of non-identical amino acids as well as sequences of identical amino acids.

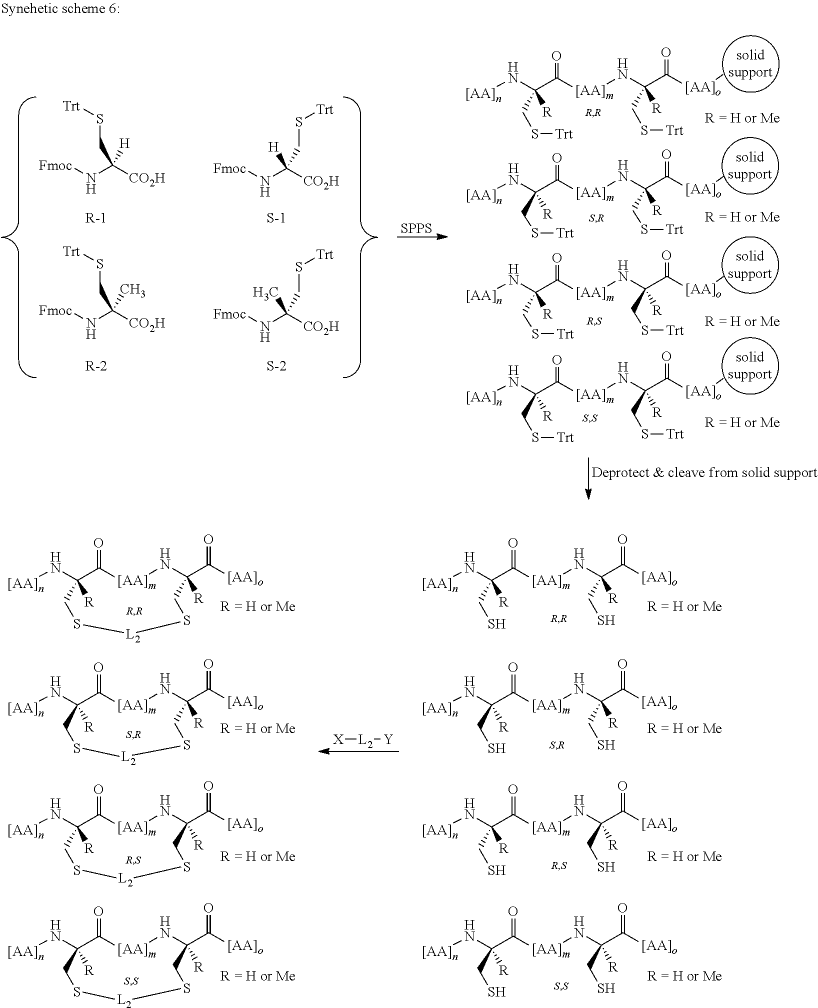

In Scheme 6, the peptidomimetic precursor contains two —SH moieties and is synthesized by solid-phase peptide synthesis (SPPS) using commercially available N-α-Fmoc amino acids such as N-α-Fmoc-S-trityl-L-cysteine or N-α-Fmoc-S-trityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-trityl monomers by known methods (“Bioorganic Chemistry: Peptides and Proteins”, Oxford University Press, New York: 1998, the entire contents of which are incorporated herein by reference). The precursor peptidomimetic is then deprotected and cleaved from the solid-phase resin by standard conditions (e.g., strong acid such as 95% TFA). The precursor peptidomimetic is reacted as a crude mixture or is purified prior to reaction with X-L₂-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6M guanidinium HCL, pH 8 (Brunel et al. (2005), Chem. Commun. (20):2552-2554). In other embodiments, the solvent used for the alkylation reaction is DMF or dichloroethane.

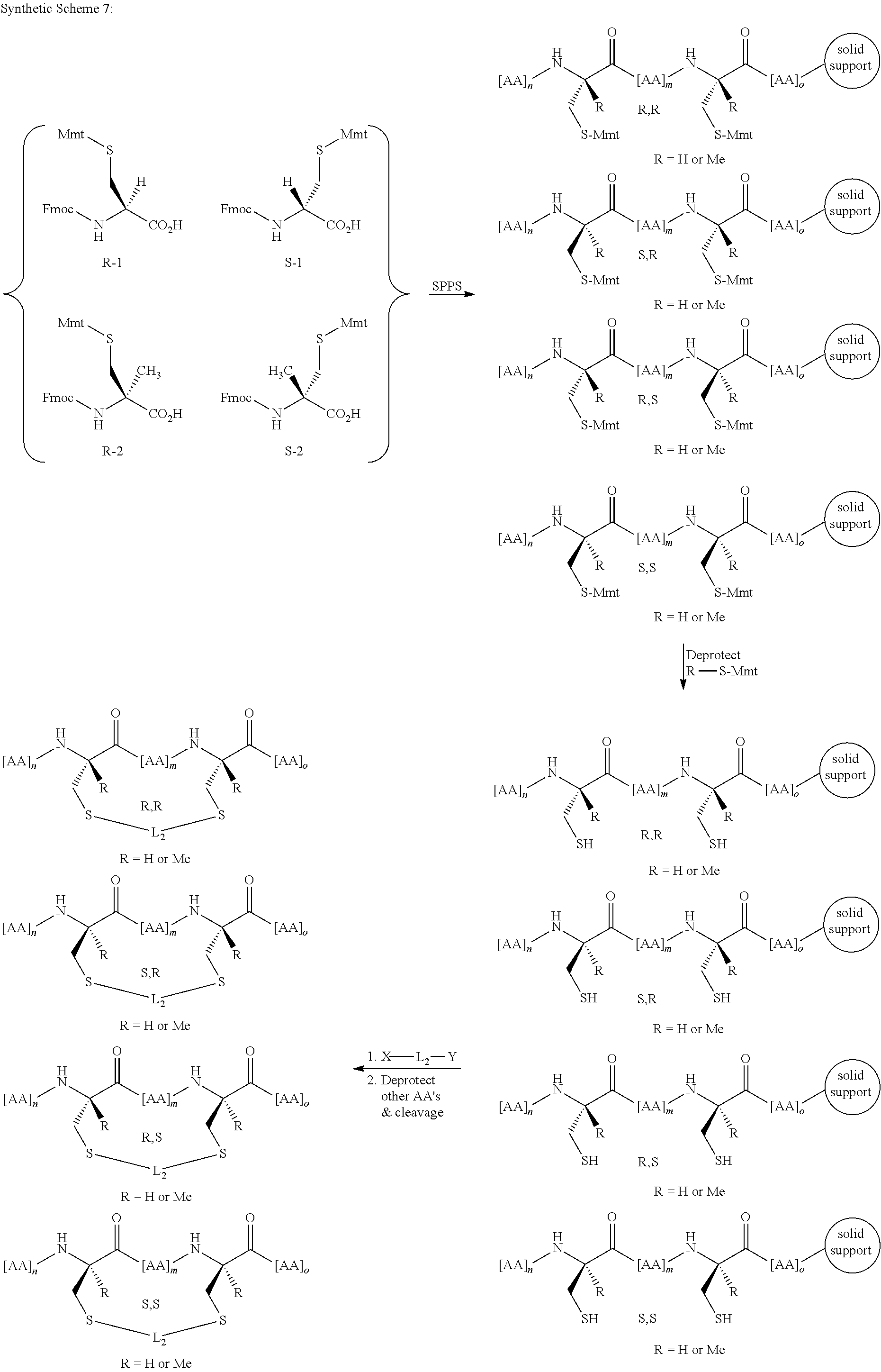

In Scheme 7, the precursor peptidomimetic contains two or more —SH moieties, of which two are specially protected to allow their selective deprotection and subsequent alkylation for macrocycle formation. The precursor peptidomimetic is synthesized by solid-phase peptide synthesis (SPPS) using commercially available N-α-Fmoc amino acids such as N-α-Fmoc-S-p-methoxytrityl-L-cysteine or N-α-Fmoc-S-p-methoxytrityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl monomers by known methods (Bioorganic Chemistry: Peptides and Proteins, Oxford University Press, New York: 1998, the entire contents of which are incorporated herein by reference). The Mmt protecting groups of the peptidomimetic precursor are then selectively cleaved by standard conditions (e.g., mild acid such as 1% TFA in DCM). The precursor peptidomimetic is then reacted on the resin with X-L₂-Y in an organic solution. For example, the reaction takes place in the presence of a hindered base such as diisopropylethylamine. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH or NH₃/DMF (Or et al. (1991), J. Org. Chem. 56:3146-3149). In other embodiments, the alkylation reaction is performed in DMF or dichloroethane. The peptidomimetic macrocycle is then deprotected and cleaved from the solid-phase resin by standard conditions (e.g., strong acid such as 95% TFA).

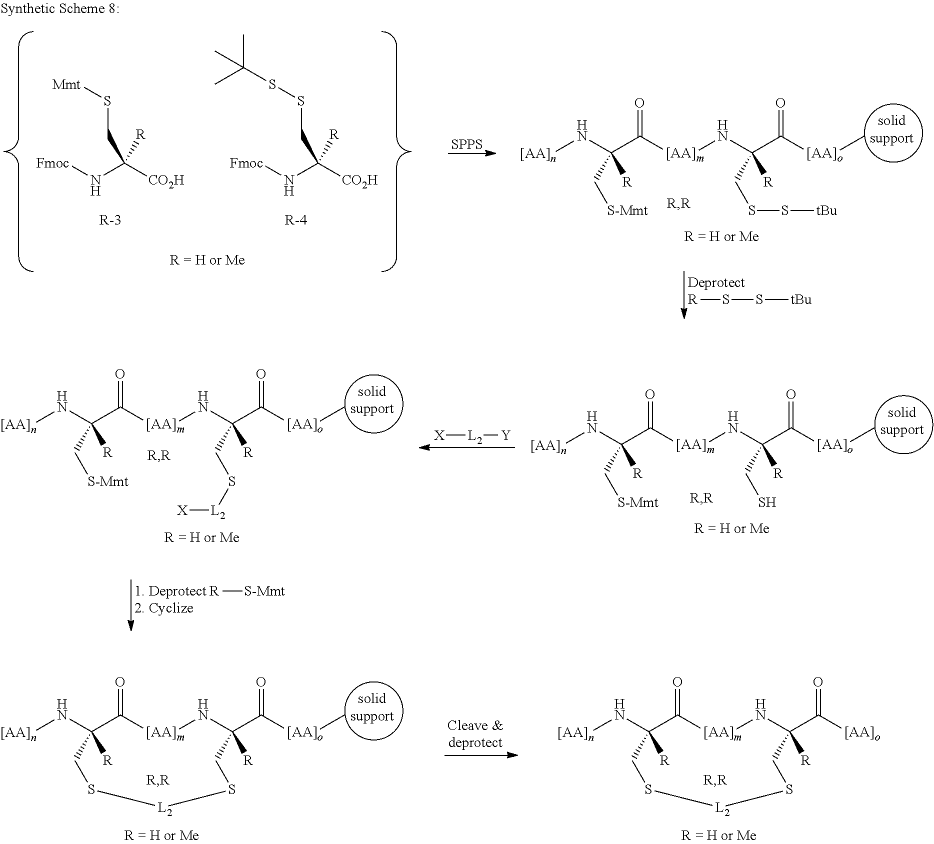

In Scheme 8, the peptidomimetic precursor contains two or more —SH moieties, of which two are specially protected to allow their selective deprotection and subsequent alkylation for macrocycle formation. The peptidomimetic precursor is synthesized by solid-phase peptide synthesis (SPPS) using commercially available N-α-Fmoc amino acids such as N-α-Fmoc-S-p-methoxytrityl-L-cysteine, N-α-Fmoc-S-p-methoxytrityl-D-cysteine, N-α-Fmoc-S—S-t-butyl-L-cysteine, and N-α-Fmoc-S—S-t-butyl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl or N-α-Fmoc-S—S-t-butyl monomers by known methods (Bioorganic Chemistry: Peptides and Proteins, Oxford University Press, New York: 1998, the entire contents of which are incorporated herein by reference). The S—S-tButyl protecting group of the peptidomimetic precursor is selectively cleaved by known conditions (e.g., 20% 2-mercaptoethanol in DMF, reference: Galande et al. (2005), J. Comb. Chem. 7:174-177). The precursor peptidomimetic is then reacted on the resin with a molar excess of X-L₂-Y in an organic solution. For example, the reaction takes place in the presence of a hindered base such as diisopropylethylamine. The Mmt protecting group of the peptidomimetic precursor is then selectively cleaved by standard conditions (e.g., mild acid such as 1% TFA in DCM). The peptidomimetic precursor is then cyclized on the resin by treatment with a hindered base in organic solutions. In some embodiments, the alkylation reaction is performed in organic solutions such as NH₃/MeOH or NH₃/DMF (Or et al. (1991), J. Org. Chem. 56:3146-3149). The peptidomimetic macrocycle is then deprotected and cleaved from the solid-phase resin by standard conditions (e.g., strong acid such as 95% TFA).

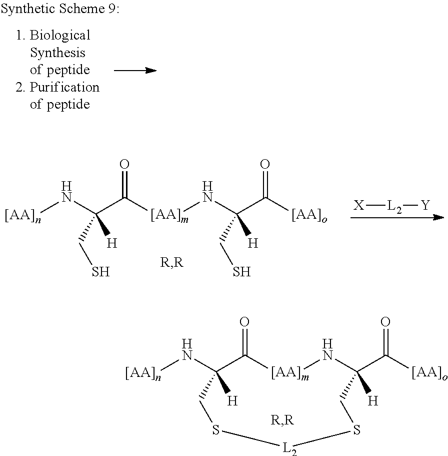

In Scheme 9, the peptidomimetic precursor contains two L-cysteine moieties. The peptidomimetic precursor is synthesized by known biological expression systems in living cells or by known in vitro, cell-free, expression methods. The precursor peptidomimetic is reacted as a crude mixture or is purified prior to reaction with X-L2-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6M guanidinium HCL, pH 8 (Brunel et al. (2005), J. Chem. Commun. (20):2552-2554). In other embodiments, the alkylation is performed in DMF or dichloroethane. In another embodiment, the alkylation is performed in non-denaturing aqueous solutions, and in yet another embodiment the alkylation is performed under conditions that favor α-helical structure formation. In yet another embodiment, the alkylation is performed under conditions that favor the binding of the precursor peptidomimetic to another protein, so as to induce the formation of the bound α-helical conformation during the alkylation.

Various embodiments for X and Y are envisioned which are suitable for reacting with thiol groups. In general, each X or Y is independently be selected from the general category shown in Table 5. For example, X and Y are halides such as —Cl, —Br or —I. Any of the macrocycle-forming linkers described herein may be used in any combination with any of the sequences shown in Tables 1-4 and also with any of the R-substituents indicated herein.

TABLE 3
Examples of Reactive Groups Capable of
Reacting with Thiol Groups and Resulting Linkages

| | Resulting |
|---|---|
| X or Y | Covalent Linkage |
| acrylamide | Thioether |
| halide (e.g. alkyl or aryl halide) | Thioether |
| sulfonate | Thioether |
| aziridine | Thioether |
| epoxide | Thioether |
| haloacetamide | Thioether |
| maleimide | Thioether |
| sulfonate ester | Thioether |

The present invention contemplates the use of both naturally-occurring and non-naturally-occurring amino acids and amino acid analogs in the synthesis of the peptidomimetic macrocycles of Formula (III). Any amino acid or amino acid analog amenable to the synthetic methods employed for the synthesis of stable bis-sulfhydryl containing peptidomimetic macrocycles can be used in the present invention. For example, cysteine is contemplated as a useful amino acid in the present invention. However, sulfur containing amino acids other than cysteine that contain a different amino acid side chain are also useful. For example, cysteine contains one methylene unit between the α-carbon of the amino acid and the terminal —SH of the amino acid side chain. The invention also contemplates the use of amino acids with multiple methylene units between the α-carbon and the terminal —SH. Non-limiting examples include α-methyl-L-homocysteine and α-methyl-D-homocysteine. In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-cysteine and α-methyl-D-cysteine.

The invention includes macrocycles in which macrocycle-forming linkers are used to link two or more —SH moieties in the peptidomimetic precursors to form the peptidomimetic macrocycles of the invention. As described above, the macrocycle-forming linkers impart conformational rigidity, increased metabolic stability and/or increased cell penetrability. Furthermore, in some embodiments, the macrocycle-forming linkages stabilize the α-helical secondary structure of the peptidomimetic macrocyles. The macrocycle-forming linkers are of the formula X-L₂-Y, wherein both X and Y are the same or different moieties, as defined above. Both X and Y have the chemical characteristics that allow one macrocycle-forming linker -L₂- to bis alkylate the bis-sulfhydryl containing peptidomimetic precursor. As defined above, the linker -L₂- includes alkylene, alkenylene, alkynylene, heteroalkylene, cycloalkylene, heterocycloalkylene, cycloarylene, or heterocycloarylene, or —R₄—K—R₄—, all of which can be optionally substituted with an R₅ group, as defined above. Furthermore, one to three carbon atoms within the macrocycle-forming linkers other than the carbons attached to the —SH of the sulfhydryl containing amino acid, are optionally substituted with a heteroatom such as N, S or O.

The L₂ component of the macrocycle-forming linker X-L₂-Y may be varied in length depending on, among other things, the distance between the positions of the two amino acid analogs used to form the peptidomimetic macrocycle. Furthermore, as the lengths of L₁ and/or L₃ components of the macrocycle-forming linker are varied, the length of L₂ can also be varied in order to create a linker of appropriate overall length for forming a stable peptidomimetic macrocycle. For example, if the amino acid analogs used are varied by adding an additional methylene unit to each of L₁ and L₃, the length of L₂ are decreased in length by the equivalent of approximately two methylene units to compensate for the increased lengths of L₁ and L₃.

In some embodiments, L₂ is an alkylene group of the formula —(CH₂)ₙ—, where n is an integer between about 1 and about 15. For example, n is 1, 2, 3, 4, 5, 6, 7, 8, 9 or 10. In other embodiments, L₂ is an alkenylene group. In still other embodiments, L₂ is an aryl group.

Table 4 shows additional embodiments of groups.

TABLE 4
Exemplary X—L₂—Y groups of the invention.

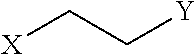

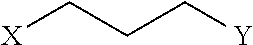

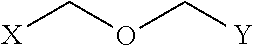

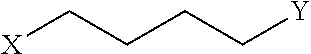

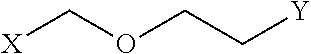

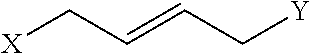

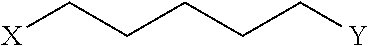

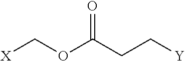

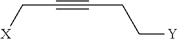

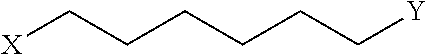

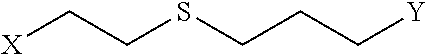

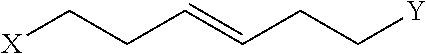

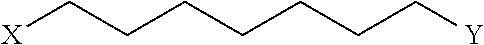

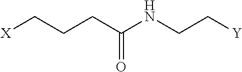

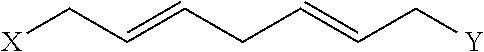

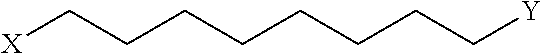

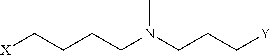

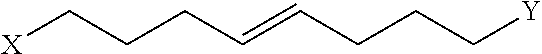

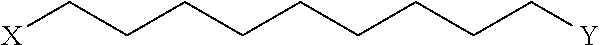

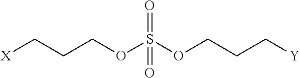

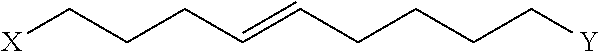

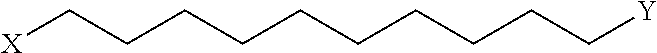

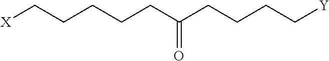

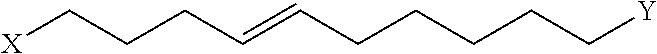

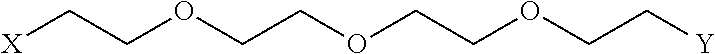

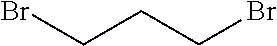

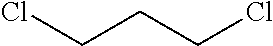

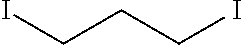

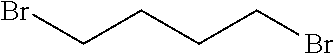

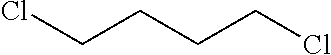

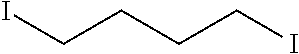

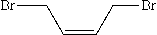

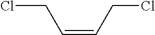

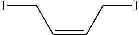

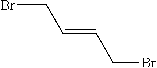

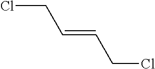

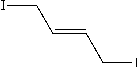

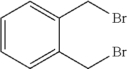

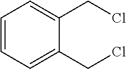

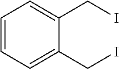

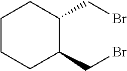

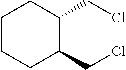

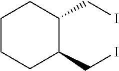

Each X and Y in this table, is, for example, independently Cl—, Br— or I—.

Additional methods of forming peptidomimetic macrocycles which are envisioned as suitable to perform the present invention include those disclosed by Mustapa, M. Firouz Mohd et al., J. Org. Chem. (2003), 68, pp. 8193-8198; Yang, Bin et al. Bioorg Med. Chem. Lett. (2004), 14, pp. 1403-1406; U.S. Pat. No. 5,364,851; U.S. Pat. No. 5,446,128; U.S. Pat. No. 5,824,483; U.S. Pat. No. 6,713,280; and U.S. Pat. No. 7,202,332. In such embodiments, aminoacid precursors are used containing an additional substituent R— at the alpha position. Such aminoacids are incorporated into the macrocycle precursor at the desired positions, which may be at the positions where the crosslinker is substituted or, alternatively, elsewhere in the sequence of the macrocycle precursor. Cyclization of the precursor is then effected according to the indicated method.

### Assays

The properties of the peptidomimetic macrocycles of the invention are assayed, for example, by using the methods described below. In some embodiments, a peptidomimetic macrocycle of the invention has improved biological properties relative to a corresponding polypeptide lacking the substituents described herein.

### Assay to Determine α-Helicity.

In solution, the secondary structure of polypeptides with α-helical domains will reach a dynamic equilibrium between random coil structures and α-helical structures, often expressed as a “percent helicity”. Thus, for example, unmodified α-helical domains may be predominantly random coils in solution, with α-helical content under 25%. Peptidomimetic macrocycles with optimized linkers, on the other hand, possess, for example, an alpha-helicity that is at least two-fold greater than that of a corresponding uncrosslinked polypeptide. In some embodiments, macrocycles of the invention will possess an alpha-helicity of greater than 50%. To assay the helicity of peptidomimetic macrocyles of the invention, such as hMAML domain-based macrocycles, the compounds are dissolved in an aqueous solution (e.g. 50 mM potassium phosphate solution at pH 7, or distilled H₂O, to concentrations of 25-50 μM). Circular dichroism (CD) spectra are obtained on a spectropolarimeter (e.g., Jasco J-710) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g. [Φ]222obs) by the reported value for a model helical decapeptide (Yang et al. (1986), Methods Enzymol. 130:208)).

### Assay to Determine Melting Temperature (Tm).

A peptidomimetic macrocycle of the invention comprising a secondary structure such as an α-helix exhibits, for example, a higher melting temperature than a corresponding uncrosslinked polypeptide. Typically peptidomimetic macrocycles of the invention exhibit Tm of >60° C. representing a highly stable structure in aqueous solutions. To assay the effect of macrocycle formation on melting temperature, peptidomimetic macrocycles or unmodified peptides are dissolved in distilled H₂O (e.g. at a final concentration of 50 μM) and the Tm is determined by measuring the change in ellipticity over a temperature range (e.g. 4 to 95° C.) on a spectropolarimeter (e.g., Jasco J-710) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

### Protease Resistance Assay.

The amide bond of the peptide backbone is susceptible to hydrolysis by proteases, thereby rendering peptidic compounds vulnerable to rapid degradation in vivo. Peptide helix formation, however, typically buries the amide backbone and therefore may shield it from proteolytic cleavage. The peptidomimetic macrocycles of the present invention may be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 μm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time (k=−1Xslope).

### Ex Vivo Stability Assay.

Peptidomimetic macrocycles with optimized linkers possess, for example, an ex vivo half-life that is at least two-fold greater than that of a corresponding uncrosslinked polypeptide, and possess an ex vivo half-life of 12 hours or more. For ex vivo serum stability studies, a variety of assays may be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure may be used: The samples are extracted by transferring 100 μl of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis.

### In Vitro Binding Assays.

To assess the binding and affinity of peptidomimetic macrocycles and peptidomimetic precursors to acceptor proteins, a fluorescence polarization assay (FPA) issued, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

For example, fluoresceinated peptidomimetic macrocycles (25 nM) are incubated with the acceptor protein (25-1000 nM) in binding buffer (140 mM NaCl, 50 mM Tris-HCL, pH 7.4) for 30 minutes at room temperature. Binding activity is measured, for example, by fluorescence polarization on a luminescence spectrophotometer (e.g. Perkin-Elmer LS50B). Kd values may be determined by nonlinear regression analysis using, for example, Graphpad Prism software (GraphPad Software, Inc., San Diego, Calif.). A peptidomimetic macrocycle of the invention shows, in some instances, similar or lower Kd than a corresponding uncrosslinked polypeptide.

### In Vitro Displacement Assays to Characterize Antagonists of Peptide-Protein Interactions.

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, for example, by fluorescence polarization on a luminescence spectrophotometer (e.g. Perkin-Elmer LS50B). Kd values may be determined by nonlinear regression analysis using, for example, Graphpad Prism software (GraphPad Software, Inc., San Diego, Calif.).

Any class of molecule, such as small organic molecules, peptides, oligonucleotides or proteins can be examined as putative antagonists in this assay.

### Binding Assays in Intact Cells.

It is possible to measure binding of peptides or peptidomimetic macrocycles to their natural acceptors in intact cells by immunoprecipitation experiments. For example, intact cells are incubated with fluoresceinated (FITC-labeled) compounds for 4 hrs in the absence of serum, followed by serum replacement and further incubation that ranges from 4-18 hrs. Cells are then pelleted and incubated in lysis buffer (50 mM Tris [pH 7.6], 150 mM NaCl, 1% CHAPS and protease inhibitor cocktail) for 10 minutes at 4° C. Extracts are centrifuged at 14,000 rpm for 15 minutes and supernatants collected and incubated with 10 μl goat anti-FITC antibody for 2 hrs, rotating at 4° C. followed by further 2 hrs incubation at 4° C. with protein A/G Sepharose (50 μl of 50% bead slurry). After quick centrifugation, the pellets are washed in lysis buffer containing increasing salt concentration (e.g., 150, 300, 500 mM). The beads are then re-equilibrated at 150 mM NaCl before addition of SDS-containing sample buffer and boiling. After centrifugation, the supernatants are optionally electrophoresed using 4%-12% gradient Bis-Tris gels followed by transfer into Immobilon-P membranes. After blocking, blots are optionally incubated with an antibody that detects FITC and also with one or more antibodies that detect proteins that bind to the peptidomimetic macrocycle.

### Cellular Penetrability Assays.

A peptidomimetic macrocycle is, for example, more cell penetrable compared to a corresponding uncrosslinked macrocycle. Peptidomimetic macrocycles with optimized linkers possess, for example, cell penetrability that is at least two-fold greater than a corresponding uncrosslinked macrocycle, and often 20% or more of the applied peptidomimetic macrocycle will be observed to have penetrated the cell after 4 hours. To measure the cell penetrability of peptidomimetic macrocycles and corresponding uncrosslinked macrocycle, intact cells are incubated with fluoresceinated peptidomimetic macrocycles or corresponding uncrosslinked macrocycle (10 μM) for 4 hrs in serum free media at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan® HCS Reader.

### Cellular Efficacy Assays.

The efficacy of certain peptidomimetic macrocycles is determined, for example, in cell-based killing assays using a variety of tumorigenic and non-tumorigenic cell lines and primary cells derived from human or mouse cell populations. Cell viability is monitored, for example, over 24-96 hrs of incubation with peptidomimetic macrocycles (0.5 to 50 μM) to identify those that kill at EC50<10 μM. Several standard assays that measure cell viability are commercially available and are optionally used to assess the efficacy of the peptidomimetic macrocycles. In addition, assays that measure Annexin V and caspase activation are optionally used to assess whether the peptidomimetic macrocycles kill cells by activating the apoptotic machinery. For example, the Cell Titer-glo assay is used which determines cell viability as a function of intracellular ATP concentration.

### In Vivo Stability Assay.

To investigate the in vivo stability of the peptidomimetic macrocycles, the compounds are, for example, administered to mice and/or rats by IV, IP, PO or inhalation routes at concentrations ranging from 0.1 to 50 mg/kg and blood specimens withdrawn at 0′, 5′, 15′, 30′, 1 hr, 4 hrs, 8 hrs and 24 hours post-injection. Levels of intact compound in 25 μL of fresh serum are then measured by LC-MS/MS as above.

### In Vivo Efficacy in Animal Models.

To determine the anti-oncogenic activity of peptidomimetic macrocycles of the invention in vivo, the compounds are, for example, given alone (IP, IV, PO, by inhalation or nasal routes) or in combination with sub-optimal doses of relevant chemotherapy (e.g., cyclophosphamide, doxorubicin, etoposide). In one example, 5×10⁶ RS4; 11 cells (established from the bone marrow of a patient with acute lymphoblastic leukemia) that stably express luciferase are injected by tail vein in NOD-SCID mice 3 hrs after they have been subjected to total body irradiation. If left untreated, this form of leukemia is fatal in 3 weeks in this model. The leukemia is readily monitored, for example, by injecting the mice with D-luciferin (60 mg/kg) and imaging the anesthetized animals (e.g., Xenogen In Vivo Imaging System, Caliper Life Sciences, Hopkinton, Mass.). Total body bioluminescence is quantified by integration of photonic flux (photons/sec) by Living Image Software (Caliper Life Sciences, Hopkinton, Mass.). Peptidomimetic macrocycles alone or in combination with sub-optimal doses of relevant chemotherapeutics agents are, for example, administered to leukemic mice (10 days after injection/day 1 of experiment, in bioluminescence range of 14-16) by tail vein or IP routes at doses ranging from 0.1 mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma, that stably expresses luciferase. These in vivo tests optionally generate preliminary pharmacokinetic, pharmacodynamic and toxicology data.

### Clinical Trials.

To determine the suitability of the peptidomimetic macrocycles of the invention for treatment of humans, clinical trials are performed. For example, patients diagnosed with cancer and in need of treatment are selected and separated in treatment and one or more control groups, wherein the treatment group is administered a peptidomimetic macrocycle of the invention, while the control groups receive a placebo or a known anti-cancer drug. The treatment safety and efficacy of the peptidomimetic macrocycles of the invention can thus be evaluated by performing comparisons of the patient groups with respect to factors such as survival and quality-of-life. In this example, the patient group treated with a peptidomimetic macrocyle show improved long-term survival compared to a patient control group treated with a placebo.

### Pharmaceutical Compositions and Routes of Administration

The peptidomimetic macrocycles of the invention also include pharmaceutically acceptable derivatives or prodrugs thereof. A “pharmaceutically acceptable derivative” means any pharmaceutically acceptable salt, ester, salt of an ester, pro-drug or other derivative of a compound of this invention which, upon administration to a recipient, is capable of providing (directly or indirectly) a compound of this invention. Particularly favored pharmaceutically acceptable derivatives are those that increase the bioavailability of the compounds of the invention when administered to a mammal (e.g., by increasing absorption into the blood of an orally administered compound) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

In some embodiments, the peptidomimetic macrocycles of the invention are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

When the compositions of this invention comprise a combination of a peptidomimetic macrocycle and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from the compounds of this invention. Alternatively, those agents are part of a single dosage form, mixed together with the compounds of this invention in a single composition.

### Methods of Use

In one aspect, the present invention provides novel peptidomimetic macrocycles that are useful in competitive binding assays to identify agents which bind to the natural ligand(s) of the proteins or peptides upon which the peptidomimetic macrocycles are modeled. For example, in the MAML/Notch/CSL system, labeled peptidomimetic macrocycles based on MAML can be used in a Notch/CSL binding assay along with small molecules that competitively bind to Notch/CSL. Competitive binding studies allow for rapid in vitro evaluation and determination of drug candidates specific for the MAML/Notch/CSL system. Such binding studies may be performed with any of the peptidomimetic macrocycles disclosed herein and their binding partners.

The invention further provides for the generation of antibodies against the peptidomimetic macrocycles. In some embodiments, these antibodies specifically bind both the peptidomimetic macrocycle and the precursor peptides, such as MAML, to which the peptidomimetic macrocycles are related. Such antibodies, for example, disrupt the native protein-protein interaction, for example, binding between MAML and Notch/CSL.

In other aspects, the present invention provides for both prophylactic and therapeutic methods of treating a subject at risk of (or susceptible to) a disorder or having a disorder associated with aberrant (e.g., insufficient or excessive) expression or activity of the molecules including Notch.

In another embodiment, a disorder is caused, at least in part, by an abnormal level of Notch or Notch ICN, (e.g., over or under expression), or by the presence of Notch exhibiting abnormal activity. As such, the reduction in the level and/or activity of the Notch or Notch ICN, or the enhancement of the level and/or activity of Notch or Notch ICN, by peptidomimetic macrocycles derived from a MAML family protein, is used, for example, to ameliorate or reduce the adverse symptoms of the disorder.

In another aspect, the present invention provides methods for treating or preventing a disease including hyperproliferative disease and inflammatory disorder by interfering with the interaction or binding between binding partners, for example, between MAML and Notch/CSL. The present invention provides for both prophylactic and therapeutic methods of treating a subject at risk of (or susceptible to) a disorder or having a disorder associated with aberrant (e.g., excessive) Notch activity. This is because the MAML peptidomimetic macrocycles are expected to act as dominant negative inhibitors of Notch/CSL activity. These methods comprise administering an effective amount of a compound of the invention to a warm blooded animal, including a human. In some embodiments, the administration of the compounds of the present invention induces cell growth arrest or apoptosis.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

In some embodiments, the peptidomimetics macrocycles of the invention is used to treat, prevent, and/or diagnose cancers and neoplastic conditions. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, the peptidomimetics macrocycles are novel therapeutic agents for controlling breast cancer, ovarian cancer, colon cancer, lung cancer, metastasis of such cancers and the like.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991), Crit. Rev. Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, the comafibromas, androblastomas, hill cell tumors, and gonadoblastoma; and metastatic tumors such as Krukenberg tumors.

In other or further embodiments, the peptidomimetics macrocycles described herein are used to treat, prevent or diagnose conditions characterized by overactive cell death or cellular death due to physiologic insult, etc. Some examples of conditions characterized by premature or unwanted cell death are or alternatively unwanted or excessive cellular proliferation include, but are not limited to hypocellular/hypoplastic, acellular/aplastic, or hypercellular/hyperplastic conditions. Some examples include hematologic disorders including but not limited to fanconi anemia, aplastic anemia, thalaessemia, congenital neutropenia, and myelodysplasia.

In other or further embodiments, the peptidomimetics macrocycles of the invention that act to decrease apoptosis are used to treat disorders associated with an undesirable level of cell death. Thus, in some embodiments, the anti-apoptotic peptidomimetics macrocycles of the invention are used to treat disorders such as those that lead to cell death associated with viral infection, e.g., infection associated with infection with human immunodeficiency virus (HIV). A wide variety of neurological diseases are characterized by the gradual loss of specific sets of neurons. One example is Alzheimer's disease (AD). Alzheimer's disease is characterized by loss of neurons and synapses in the cerebral cortex and certain subcortical regions. This loss results in gross atrophy of the affected regions. Both amyloid plaques and neurofibrillary tangles are visible in brains of those afflicted by AD. Alzheimer's disease has been identified as a protein misfolding disease, due to the accumulation of abnormally folded A-beta and tau proteins in the brain. Plaques are made up of β-amyloid. β-amyloid is a fragment from a larger protein called amyloid precursor protein (APP). APP is critical to neuron growth, survival and post-injury repair. In AD, an unknown process causes APP to be cleaved into smaller fragments by enzymes through proteolysis. One of these fragments is fibrils of β-amyloid, which form clumps that deposit outside neurons in dense formations known as senile plaques. Plaques continue to grow into insoluble twisted fibers within the nerve cell, often called tangles. Disruption of the interaction between β-amyloid and its native receptor is therefore important in the treatment of AD. The anti-apoptotic peptidomimetics macrocycles of the invention are used, in some embodiments, in the treatment of AD and other neurological disorders associated with cell apoptosis. Such neurological disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis. In other or further embodiments, the anti-apoptotic peptidomimetics macrocycles of the invention are used to treat all such disorders associated with undesirable cell death.

Some examples of neurologic disorders that are treated with the peptidomimetics macrocycles described herein include but are not limited to Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann Straussler-Scheinker Syndrome, Bovine Spongiform Encephalitis, a prion-mediated disease, and Huntington's Disease.

In another embodiment, the peptidomimetics macrocycles described herein are used to treat, prevent or diagnose inflammatory disorders. Numerous types of inflammatory disorders exist. Certain inflammatory diseases are associated with the immune system, for example, autoimmune diseases. Autoimmune diseases arise from an overactive immune response of the body against substances and tissues normally present in the body, i.e. self antigens. In other words, the immune system attacks its own cells. Autoimmune diseases are a major cause of immune-mediated diseases. Rheumatoid arthritis is an example of an autoimmune disease, in which the immune system attacks the joints, where it causes inflammation (i.e. arthritis) and destruction. It can also damage some organs, such as the lungs and skin. Rheumatoid arthritis can lead to substantial loss of functioning and mobility. Rheumatoid arthritis is diagnosed with blood tests especially the rheumatoid factor test. Some examples of autoimmune diseases that are treated with the peptidomimetics macrocycles described herein include, but are not limited to, acute disseminated encephalomyelitis (ADEM), Addison's disease, ankylosing spondylitis, antiphospholipid antibody syndrome (APS), autoimmune hemolytic anemia, autoimmune hepatitis, autoimmune inner ear disease, Bechet's disease, bullous pemphigoid, coeliac disease, Chagas disease, Churg-Strauss syndrome, chronic obstructive pulmonary disease (COPD), Crohn's disease, dermatomyositis, diabetes mellitus type 1, endometriosis, Goodpasture's syndrome, Graves' disease, Guillain-Barré syndrome (GBS), Hashimoto's disease, Hidradenitis suppurativa, idiopathic thrombocytopenic purpura, inflammatory bowl disease (IBD), interstitial cystitis, lupus erythematosus, morphea, multiple sclerosis, myasthenia gravis, narcolepsy, neuromyotonia, pemphigus vulgaris, pernicious anaemia, Polymyositis, polymyalgia rheumatica, primary biliary cirrhosis, psoriasis, rheumatoid arthritis, schizophrenia, scleroderma, Sjögren's syndrome, temporal arteritis (also known as “giant cell arteritis”), Takayasu's arteritis, Vasculitis, Vitiligo, and Wegener's granulomatosis.

Some examples of other types of inflammatory disorders that are treated with the peptidomimetics macrocycles described herein include, but are not limited to, allergy including allergic rhinitis/sinusitis, skin allergies (urticaria/hives, angioedema, atopic dermatitis), food allergies, drug allergies, insect allergies, and rare allergic disorders such as mastocytosis, asthma, arthritis including osteoarthritis, rheumatoid arthritis, and spondyloarthropathies, primary angitis of the CNS, sarcoidosis, organ transplant rejection, fibromyalgia, fibrosis, pancreatitis, and pelvic inflammatory disease.

Examples of cardiovascular disorders (e.g., inflammatory disorders) that are treated or prevented with the peptidomimetics macrocycles of the invention include, but are not limited to, aortic valve stenosis, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve or other implantable devices. Preferred cardiovascular disorders include atherosclerosis, myocardial infarction, aneurism, and stroke.

### Example 1

### Design of Peptidomimetic Macrocycle Inhibitors of Notch Signalling Based on hMAML

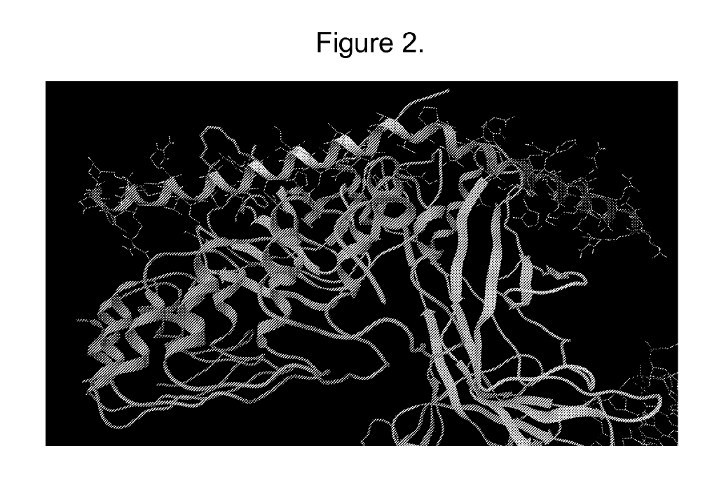

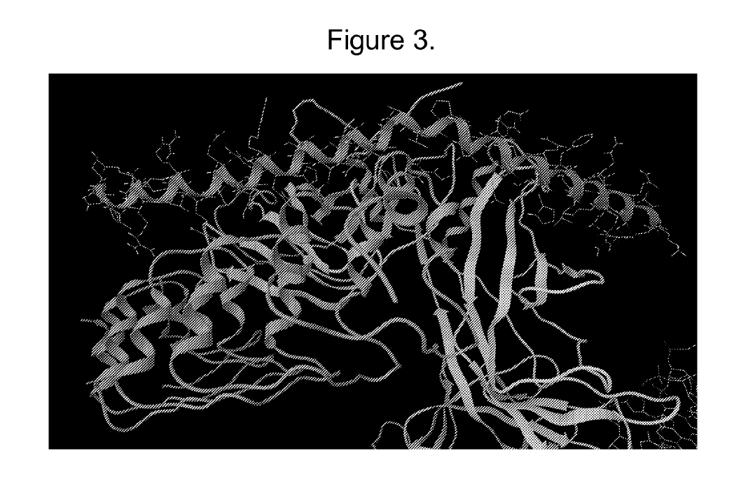

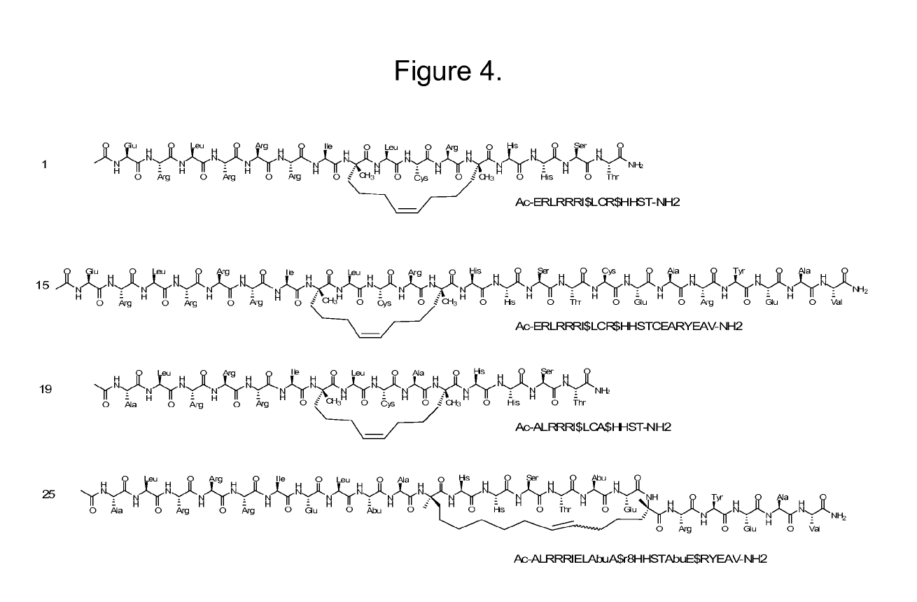

The binding of uncrosslinked polypeptides (FIG. 1) and the corresponding peptidomimetic macrocycles (FIGS. 2 and 3) of the invention to Notch/CSL/DNA complex was studied by modeling experiments. The sequence of the corresponding uncrosslinked polypeptide was ERLRRRIELCRRHHSTCEARYE (residues 21-42 of hMAML). Solvent exposed side-chains available for cross-linking are underlined, and selected side-chains available for cross-linking are shown in FIG. 1. Two embodiments representing peptidomimetic macrocycles of the invention were studied. In FIG. 2, a cis-olefin i→i+4 cross-link between Glu28 and Arg32 is shown. In FIG. 3, a cis-olefin i→i+4 cross-link between Ser35 and Ala39 is shown.

### Example 2

### Synthesis of Peptidomimetic Macrocycles of Formula (I)

α-helical crosslinked polypeptides are synthesized, purified and analyzed as previously described (Schafineister et al. (2000), J. Am. Chem. Soc. 122:5891-5892; Walensky et al (2004) Science 305:1466-70; Walensky et al (2006) Mol Cell 24:199-210) and as indicated below. The following macrocycles derived from the human MAML peptide sequences are used in this study:

| | | Calculated m/z | Calculated | Calculated | Observed |
|---|---|---|---|---|---|
| Compound # | Sequence | (M + H) | m/z (M + 2H) | m/z (M + 3H) | m/z |
| 1 | Ac-ERLRRRI$LCR$HHST-NH2 | 2124.21 | 1063.11 | 709.08 | 708.72 |
| 2 | Ac-ERLRRRIELCRRHHST-NH2 | 2159.19 | 1080.60 | 720.74 | 720.39 |
| 3 | Ac-ERLARAI$LCR$HHST-NH2 | 1954.08 | 978.05 | 652.37 | 652.11 |
| 4 | Ac-ERLRRRI$LAR$HHST-NH2 | 2092.24 | 1047.13 | 698.42 | 698.09 |
| 5 | Ac-ERLARAI$LAR$HHST-NH2 | 1922.11 | 962.06 | 641.71 | 641.42 |
| 6 | Ac-ERLRR$IEL$RAHHST-NH2 | 2065.18 | 1033.60 | 689.40 | 689.01 |
| 7 | Ac-EALRRRI$LCA$HHST-NH2 | 1954.08 | 978.05 | 652.37 | 652.11 |
| 8 | Ac-REL$RRI$LCRRHHST-NH2 | 2124.21 | 1063.11 | 709.08 | 709.05 |
| 9 | Ac-RRIELCRRHHSTCEARYEAV-NH2 | 2526.26 | 1264.14 | 843.09 | 842.92 |
| 10 | Ac-RRIELCRRHH$/TCE$/RYEAV-NH2 | 2646.39 | 1324.20 | 883.14 | 882.96 |
| 11 | Ac-RAIELCRAHH$TCE$RYEAV-NH2 | 2448.23 | 1225.12 | 817.08 | 816.70 |
| 12 | Ac-RRIELCRAHH$TCE$RYEAV-NH2 | 2533.3 | 1267.66 | 845.44 | 845.07 |
| 13 | Ac-RAIELCRRHH$TCE$RYEAV-NH2 | 2533.3 | 1267.66 | 845.44 | 845.07 |
| 14 | Ac-ERLRRRIELCRRHH$TCE$RYEAV-NH2 | 3172.69 | 1587.35 | 1058.57 | 1058.17 |
| 15 | Ac-ERLRRRI$LCR$HHSTCEARYEAV-NH2 | 3045.61 | 1523.81 | 1016.21 | 1016.16 |
| 16 | Ac-ERLRR$IEL$RRHHST-NH2 | 2150.25 | 1076.13 | 717.76 | 717.45 |
| 17 | Ac-RRIELARRHH$TAE$RYEAV-NH2 | 2554.42 | 1278.22 | 852.48 | 852.41 |
| 18 | Ac-RRIELARR$HST$EARYEAV-NH2 | 2504.39 | 1253.20 | 835.80 | 835.42 |
| 19 | Ac-ALRRRI$LCA$HHST-NH2 | 1825.04 | 913.53 | 609.35 | 609.06 |
| 20 | Ac-ALRRRI$LAA$HHST-NH2 | 1793.07 | 897.54 | 598.70 | 598.41 |
| 21 | Ac-ALRRRI$LSA$HHST-NH2 | 1809.07 | 905.54 | 604.03 | 603.77 |
| 22 | Ac-ERLRRRIELAARHH$TAE$RYEAV-NH2 | 3023.68 | 1512.85 | 1008.90 | 1008.59 |
| 23 | Ac-ALRRRI$LAbuA$HHST-NH2 | 1807.09 | 904.55 | 603.37 | 603.30 |
| 24 | Ac-ALRRRIELAARHH$TAE$RYEAV-NH2 | 2809.57 | 1405.79 | 937.53 | 937.16 |
| 25 | Ac-ALRRRIELAbuA$r8HHSTAbuE$RYEAV-NH2 | 2810.58 | 1406.30 | 937.87 | 937.36 |
| 26 | Ac-ALRRRI$LAbuA$HHSTAEARYEAV-NH2 | 2696.52 | 1349.27 | 899.85 | 899.45 |
| 27 | Ac-ALRRRI$LAA$HHSTAEARYEAV-NH2 | 2682.5 | 1342.26 | 895.17 | 894.56 |
| 28 | Ac-ALRRRI$LSA$HHSTAEARYEAV-NH2 | 2698.49 | 1350.25 | 900.50 | 899.91 |
| 29 | Ac-ALRRRI$LAbuA$HHSTAbuEARYEAV-NH2 | 2710.53 | 1356.27 | 904.52 | 904.35 |
| 30 | Ac-REL$RRI$LCARHHST-NH2 | 2039.15 | 1020.58 | 680.72 | 680.28 |
| 31 | Ac-RALRRRI$LAbuA$HHST-NH2 | 1963.19 | 982.60 | 655.40 | 654.88 |
| 32 | Ac-RELRREI$LCR$HHST-NH2 | 2097.15 | 1049.58 | 700.06 | 699.83 |
| 33 | Ac-ELCRRHH$TCE$RYEAV-NH2 | 2193.07 | 1097.54 | 732.03 | 731.95 |
| 34 | Ac-ELCRRHHSTCEARYEAV-NH2 | 2100.97 | 1051.49 | 701.33 | 700.89 |
| 35 | Ac-RELRREILLCRRHHST-NH2 | 2116.17 | 1059.09 | 706.40 | 706.25 |

In the sequences above, Nle represents norleucine, Aib represents 2-aminoisobutyric acid, Abu represents (S)-2-aminobutyric acid, Ac represents N-terminal acetyl and NH2 represents C-terminal amide. The amino acid represented as $ is (S)-α-(2′-pentenyl) alanine (“S5-olefin amino acid”) and the amino acid represented as $r8 is (R)-α-(2′-octenyl) alanine (“R8 olefin amino acid”). Following incorporation of such amino acids into precursor polypeptides, the terminal olefins are reacted with a metathesis catalyst, leading to the formation of the peptidomimetic macrocycles. Macrocycles connecting two $ amino acids possess an all-carbon crosslinker comprising eight carbon atoms between the alpha carbons of each amino acid with a double bond between the fourth and fifth carbon atoms and wherein each α-carbon atom to which the crosslinker is attached is additionally substituted with a methyl group. Macrocycles connecting one $r8 amino acid to one $ amino acid possess an all-carbon crosslinker comprising eleven carbon atoms between the alpha carbons of each amino acid with a double bond between the seventh and eighth carbon atoms and wherein each α-carbon atom to which the crosslinker is attached is additionally substituted with a methyl group. If no metathesis reaction is performed, then the olefin amino acids in the resulting polypeptide are labeled as $/ and $r8/to denote an uncrosslinked peptide containing the unmodified (S)-α-(2′-pentenyl) alanine (“S5-olefin amino acid”) or the unmodified (R)-α-(2′-octenyl) alanine, respectively. Predicted and measured m/z spectra are provided.

The α,α-disubstituted amino acids and amino acid precursors disclosed in the cited references may be employed in synthesis of the peptidomimetic macrocycle precursor polypeptides. Alpha,alpha-disubstituted non-natural amino acids containing olefinic side chains are synthesized according to Williams et al. (1991) J. Am. Chem. Soc. 113:9276; and Schafineister et al. (2000) J. Am. Chem. Soc. 122:5891. Crosslinked polypeptides are designed by replacing two naturally occurring amino acids (see above) with the corresponding synthetic amino acids. Substitutions are made at i and i+4 positions and at i and i+7 positions.

The non-natural amino acids (R and S enantiomers of the 5-carbon olefinic amino acid and the S enantiomer of the 8-carbon olefinic amino acid) are characterized by nuclear magnetic resonance (NMR) spectroscopy (Varian Mercury 400) and mass spectrometry (Micromass LCT). Peptide synthesis is performed either manually or on an automated peptide synthesizer (Applied Biosystems, model 433A), using solid phase conditions, rink amide AM resin (Novabiochem), and Fmoc main-chain protecting group chemistry. For the coupling of natural Fmoc-protected amino acids (Novabiochem), 10 equivalents of amino acid and a 1:1:2 molar ratio of coupling reagents HBTU/HOBt (Novabiochem)/DIEA are employed. Non-natural amino acids (4 equiv) are coupled with a 1:1:2 molar ratio of HATU (Applied Biosystems)/HOBt/DIEA. Olefin metathesis is performed in the solid phase using 10 mM Grubbs catalyst (Blackewell et al. 1994 supra) (Materia) dissolved in degassed dichloromethane and reacted for 2 hours at room temperature. Isolation of metathesized compounds is achieved by trifluoroacetic acid-mediated deprotection and cleavage, ether precipitation to yield the crude product, and high performance liquid chromatography (HPLC) (Varian ProStar) on a reverse phase C18 column (Varian) to yield the pure compounds. Chemical composition of the pure products is confirmed by LC/MS mass spectrometry (Micromass LCT interfaced with Agilent 1100 HPLC system) and amino acid analysis (Applied Biosystems, model 420A).

### Example 3

### Cell Viability Assays of Tumor Cell Lines Treated with Peptidomimetic Macrocycles of the Invention

Molt-4 cell line (ATCC catalog #CRL-1582) was grown in specific serum-supplemented media (RPMI-1640, Invitrogen catalog #72400) as recommended by ATCC. A day prior to the initiation of the study, cells were split at optimal cell density (2×10⁵-5×10⁵ cells/ml) to assure actively dividing cells. The next day, cells were washed twice in serum-free Opti-MEM media (Invitrogen, Catalog #51985) and cells were then plated at optimal cell density (10,000 cells/well) in 50 μl Opti-MEM media or Opti-MEM supplemented with 2%, 4% or 10% human serum (Bioreclamation, catalog #HMSRM) in 96-well white tissue culture plate (Nunc, catalog #136102).

For serum free experiment, peptidomimetic macrocycles were diluted from 2 mM stocks (100% DMSO) in sterile water to prepare 400 μM working solutions. The macrocycles and controls were diluted 10-fold first and then serially two-fold diluted in Opti-MEM in dosing plates to provide concentrations of between 1.2 and 40 μM. 50 μL of each dilution was then added to the appropriate wells of the test plate to achieve final concentrations of the polypeptides equal to between 0.6 to 20 μM. For studies using Opti-MEM supplemented with human serum (Bioreclamation, catalog #HMSRM), peptidomimetic macrocycles were diluted from 10 mM stocks (100% DMSO) in sterile water to prepare 2 mM working solutions. The peptide macrocycles and controls were diluted 10-fold first and then serially two-fold diluted in Opti-MEM in the presence of 2%, 4% or 10% of human serum to provide concentrations of the polypeptides equal to between 6.25 to 200 μM in dosing plates. 50 μL of each dilution was then added to the appropriate welts of the test plate to achieve final concentrations of the polypeptides equal to between 3.125 to 100 μM. Controls included wells without polypeptides containing the same concentration of DMSO as the wells containing the macrocycles, wells containing 0.1% Triton X-100 and wells containing no cells. Plates were incubated for 48 hours at 37° C. in humidified 5% CO₂ atmosphere.

At the end of the incubation period, CellTiter-Glo assay was performed according to manufacturer's instructions (Promega, catalog #G7573) and luminescence was measured using Synergy HT Plate reader (BioTek).

The following macrocycles derived from the human MAML peptide sequences were tested in cell viability assays with the MOLT-4 tumor cell line:

| Com- | | Molt 4 |
|---|---|---|
| pound | | viability |
| # | Sequence | (EC50, uM) |
| 1 | Ac-ERLRRRI$LCR$HHST-NH2 | 3.6 |
| 2 | Ac-ERLRRRIELCRRHHST-NH2 | >20 |
| 4 | Ac-ERLRRRI$LAR$HHST-NH2 | >20 |
| 7 | Ac-EALRRRI$LCA$HHST-NH2 | 11.5 |
| 8 | Ac-REL$RRI$LCRRHHST-NH2 | 11.5 |
| 15 | Ac-ERLRRRI$LCR$HHSTCEARYEAV-NH2 | 2.2 |
| 16 | Ac-ERLRR$IEL$RRHHST-NH2 | 11.7 |
| 17 | Ac-RRIELARRHH$TAE$RYEAV-NH2 | >20 |
| 18 | Ac-RRIELARR$HST$EARYEAV-NH2 | >20 |
| 19 | Ac-ALRRRI$LCA$HHST-NH2 | 4.2 |
| 20 | Ac-ALRRRI$LAA$HHST-NH2 | 24.4 |
| 21 | Ac-ALRRRI$LSA$HHST-NH2 | >20 |
| 22 | Ac-ERLRRRIELAARHH$TAE$RYEAV-NH2 | >20 |
| 24 | Ac-ALRRRIELAARHH$TAE$RYEAV-NH2 | >20 |
| 25 | Ac-ALRRRIELAbuA$r8HHSTAbuE$RYEAV-NH2 | >20 |
| 26 | Ac-ALRRRI$LAbuA$HHSTAEARYEAV-NH2 | >20 |
| 31 | Ac-RALRRRI$LAbuA$HHST-NH2 | 3.7 |

### Example 4

### Determination of Apparent Affinity to Human Serum Proteins (Kd*)

The measurement of apparent Kd values for serum protein by EC50 shift analysis provides a simple and rapid means of quantifying the propensity of experimental compounds to bind HSA and other serum proteins. A linear relationship exists between the apparent EC50 in the presence of serum protein (EC'50) and the amount of serum protein added to an in vitro assay. This relationship is defined by the binding affinity of the compound for serum proteins, expressed as Kd*. This term is an experimentally determined, apparent dissociation constant that may result from the cumulative effects of multiple, experimentally indistinguishable, binding events. The form of this relationship is presented here in Eq. 0.3, and its derivation can be found in Copeland et al, Biorg. Med. Chem. Lett. 2004, 14:2309-2312.

$${EC}_{50}^{′} = {EC}_{50} + P ( \frac{n}{1 + \frac{{K}_{d}^{*}}{{EC}_{50}}} ) \qquad ( 0.1 )$$

A significant proportion of serum protein binding can be ascribed to drug interactions with HSA, due to the very high concentration of this protein in serum (35-50 g/L or 530-758 μM). To calculate the Kd value for these compounds we have assumed that the shift in EC50 upon protein addition can be ascribed fully to the HSA present in the added serum, where P is 700 μM for 100% serum, P is 70 μM for 10% serum, etc. We further made the simplifying assumption that all of the compounds bind HSA with a 1:1 stoichiometry, so that the term n in Eq. (0.3) is fixed at unity. With these parameters in place we calculated the Kd* value for each stapled peptide from the changes in EC50 values with increasing serum (and serum protein) concentrations by nonlinear regression analysis of Eq. 0.3 using Mathematica 4.1 (Wolfram Research, Inc., www.wolfram.com). EC'50 values in whole blood are estimated by setting P in Eq. 0.3 to 700 μM [HSA].

The free fraction in blood is estimated using the following equation, as derived by Trainor, Expert Opin. Drug Disc., 2007, 2(1):51-64, where [HSA] total is set at 700 μM.

$$FreeFraction = \frac{{K}_{d}^{*}}{{K}_{d}^{*} + {[ HSA ]}_{total}} \qquad ( 0.2 )$$

The following macrocycles derived from the human MAML peptide sequences were tested in cell viability assays (described above) with the MOLT-4 tumor cell line at a range of human serum protein concentrations to determine their apparent affinity to human serum proteins and the projected EC50s in whole human blood:

| | | | | | Free Fraction | |
|---|---|---|---|---|---|---|
| | No serum | 2% serum | 10% serum | | est. in blood, | EC50 est. in |
| Compound # | EC50, μM | EC50, μM | EC50, μM | Serum Kd* | μM | blood, μM |
| 1 | 12.0 | 55.1 | >100 | <0.1 | <0.1% | 2167 |
| 4 | >20 | >100 | >100 | <0.1 | <0.1% | >4000 |
| 19 | 2.4 | 14.7 | 57.5 | 0.6 | 0.10% | 549.4 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A peptidomimetic macrocycle comprising an amino acid sequence which is at least about 60% identical to an amino acid sequence chosen from the group consisting of the amino acid sequences in Table 1.

2. The peptidomimetic macrocycle of claim 1, wherein the amino acid sequence of said peptidomimetic macrocycle is at least about 80% identical to an amino acid sequence chosen from the group consisting of the amino acid sequences in Table 1.

3. The peptidomimetic macrocycle of claim 1, wherein the amino acid sequence of said peptidomimetic macrocycle is at least about 90% identical to an amino acid sequence chosen from the group consisting of the amino acid sequences in Table 1.

4. The peptidomimetic macrocycle of claim 1, wherein the amino acid sequence of said peptidomimetic macrocycle is chosen from the group consisting of the amino acid sequences in Table 1.

5. The peptidomimetic macrocycle of claim 1, wherein the peptidomimetic macrocycle comprises a helix.

6. The peptidomimetic macrocycle of claim 1, wherein the peptidomimetic macrocycle comprises an α-helix.

7. The peptidomimetic macrocycle of claim 1, wherein the peptidomimetic macrocycle comprises an α,α-disubstituted amino acid.

8. The peptidomimetic macrocycle of claim 1, wherein the peptidomimetic macrocycle comprises a crosslinker linking the α-positions of at least two amino acids.

9. The peptidomimetic macrocycle of claim 8, wherein at least one of said two amino acids is an α,α-disubstituted amino acid.

10. The peptidomimetic macrocycle of claim 8, wherein the peptidomimetic macrocycle has the formula: 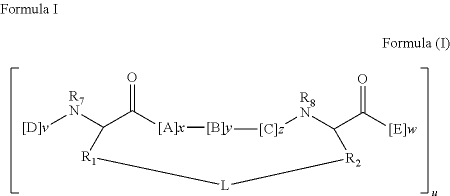
wherein:
each A, C, D, and E is independently a natural or non-natural amino acid;
B is a natural or non-natural amino acid, amino acid analog 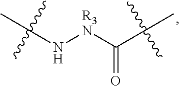
[—NH-L₃-CO—], [—NH-L₃-SO₂—], or [—NH-L₃-];
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
L is a macrocycle-forming linker of the formula -L₁-L₂-;
L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;
each R₄ is alkylene, alkenylene, alkynylene, heteroalkylene, cycloalkylene, heterocycloalkylene, arylene, or heteroarylene;
each K is O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v and w are independently integers from 1-1000;
u, x, y and z are independently integers from 0-10; and
n is an integer from 1-5.

11. The peptidomimetic macrocycle of claim 1, wherein the peptidomimetic macrocycle comprises a crosslinker linking a backbone amino group of a first amino acid to a second amino acid within the peptidomimetic macrocycle.

12. The peptidomimetic macrocycle of claim 11, wherein the peptidomimetic macrocycle has the formula (IV) or (IVa): 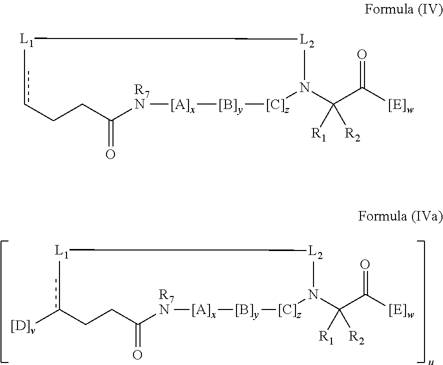
wherein:
each A, C, D, and E is independently a natural or non-natural amino acid;
B is a natural or non-natural amino acid, amino acid analog 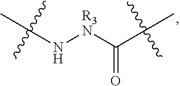
[—NH-L₃-CO—], [—NH-L₃-SO₂—], or [—NH-L₃-];
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;
each R₄ is alkylene, alkenylene, alkynylene, heteroalkylene, cycloalkylene, heterocycloalkylene, arylene, or heteroarylene;
each K is O, S, SO, SO₂, CO, CO₂, or CONR₃;
each R₅ is independently halogen, alkyl, —OR₆, —N(R₆)₂, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅;
v and w are independently integers from 1-1000;
u, x, y and z are independently integers from 0-10; and
n is an integer from 1-5.

13. A method of treating cancer in a subject comprising administering to the subject a peptidomimetic macrocycle of claim 1.

14. A method of modulating the activity of Notch in a subject comprising administering to the subject a peptidomimetic macrocycle of claim 1.

15. A method of antagonizing the interaction between MAML and Notch or CSL proteins in a subject comprising administering to the subject a peptidomimetic macrocycle of claim 1.
